SEQUENCE LISTING (141 sequences)

SEQ ID NO: 1 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 2 - protein, 447 aa, synthetic construct [sequence omitted]

SEQ ID NO: 3 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 4 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 5 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 6 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 7 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 8 - protein, 447 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFY

SEQ ID NO: 9 - protein, 447 aa, synthetic construct
LNFRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFY

SEQ ID NO: 10 - protein, 447 aa, synthetic construct
LNFRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 11 - protein, 447 aa, synthetic construct
LNFRAPPVIPNVPFLWAWNAPSEFCLGKFDEPLDMSLFSFIGSPRINATGQGVTIFHVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 12 - protein, 447 aa, synthetic construct
LNFRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 13 - protein, 447 aa, synthetic construct
LNFRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 14 - protein, 447 aa, synthetic construct
LNFRACPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFY

SEQ ID NO: 15 - protein, 447 aa, synthetic construct
LNFRAPPVIPNVPFLWAWNAPSEFCLGKFDEPLDMCLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFY

SEQ ID NO: 16 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 17 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 18 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIHGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 19 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 20 - protein, 447 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFY

SEQ ID NO: 21 - protein, 447 aa, synthetic construct
LNFRAPPVIPNVPFLWAWNAPSEFCLGKFDEPLDMCLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFY

SEQ ID NO: 22 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 23 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 24 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGRLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 25 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 26 - protein, 447 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFY

SEQ ID NO: 27 - protein, 447 aa, synthetic construct
LNFRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFY

SEQ ID NO: 28 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 29 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 30 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTKSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 31 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 32 - protein, 447 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFY

SEQ ID NO: 33 - protein, 447 aa, synthetic construct
LNFRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFY

SEQ ID NO: 34 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 35 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 36 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGALSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 37 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 38 - protein, 447 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFY

SEQ ID NO: 39 - protein, 447 aa, synthetic construct
LNFRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFY

SEQ ID NO: 40 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 41 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 42 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 43 - protein, 448 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 44 - protein, 448 aa, synthetic construct
LNFRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 45 - protein, 448 aa, synthetic construct
LNFRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 46 - protein, 448 aa, synthetic construct
LNFRAPPVIPNVPFLWAWNAPSEFCLGKFDEPLDMSLFSFIGSPRINATGQGVTIFHVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 47 - protein, 448 aa, synthetic construct
LNFRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 48 - protein, 448 aa, synthetic construct
LNFRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 49 - protein, 448 aa, synthetic construct
LNFRACPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 50 - protein, 448 aa, synthetic construct
LNFRAPPVIPNVPFLWAWNAPSEFCLGKFDEPLDMCLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 51 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 52 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 53 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIHGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 54 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 55 - protein, 448 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 56 - protein, 448 aa, synthetic construct
LNFRAPPVIPNVPFLWAWNAPSEFCLGKFDEPLDMCLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 57 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 58 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 59 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGRLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 60 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 61 - protein, 448 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 62 - protein, 448 aa, synthetic construct
LNFRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 63 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 64 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 65 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTKSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 66 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 67 - protein, 448 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 68 - protein, 448 aa, synthetic construct
LNFRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 69 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 70 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 71 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGALSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 72 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 73 - protein, 448 aa, synthetic construct
LNFRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 74 - protein, 448 aa, synthetic construct
LNFRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 75 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 76 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 77 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 78 - protein, 446 aa, synthetic construct
FRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 79 - protein, 446 aa, synthetic construct
FRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 80 - protein, 446 aa, synthetic construct
FRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 81 - protein, 446 aa, synthetic construct
FRAPPVIPNVPFLWAWNAPSEFCLGKFDEPLDMSLFSFIGSPRINATGQGVTIFHVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 82 - protein, 446 aa, synthetic construct
FRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 83 - protein, 446 aa, synthetic construct
FRAPPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 84 - protein, 446 aa, synthetic construct
FRACPVIPNVPFLWAWNAPSEFCLGKFDEPLDMSLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 85 - protein, 446 aa, synthetic construct
FRAPPVIPNVPFLWAWNAPSEFCLGKFDEPLDMCLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 86 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 87 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 88 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIHGTLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 89 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 90 - protein, 446 aa, synthetic construct
FRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 91 - protein, 446 aa, synthetic construct
FRAPPVIPNVPFLWAWNAPSEFCLGKFDEPLDMCLFSFIGSPRINATGQGVTIFHVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIHGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 92 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 93 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 94 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGRLSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 95 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 96 - protein, 446 aa, synthetic construct
FRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 97 - protein, 446 aa, synthetic construct
FRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGRLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 98 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 99 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 100 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGTKSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 101 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 102 - protein, 446 aa, synthetic construct
FRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 103 - protein, 446 aa, synthetic construct
FRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTKSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 104 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 105 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGICQKISLQDHLDKAKKDITFYMPVDNLGMAVIDWEEWRPTWARNWKPKDVYKNRSIELVQQQNVQLSLTEATEKAKQEFEKCGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 106 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFCETVALGASGIVIWGALSIMRSMKSCLLLDNYMETILNPYIINVTLAAKMCSQVLCQEQGVCIRKNWNSSDCLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 107 - protein, 446 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHCWGYYLFPDCYNHHYKKPGYNGSCFNVEIKRNDDLSWLWNESTCLYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 108 - protein, 446 aa, synthetic construct
FRACPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCCSTLSCKEKADVKDTDAVDVCIADGVCIDAFLKPPMETEEPQIFYN

SEQ ID NO: 109 - protein, 446 aa, synthetic construct
FRAPPVIPNVPFLWAWNAPSEFCLGKFDEPLDMCLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGALSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIACGVCIDAFLKPPMETEEPQIFYN

SEQ ID NO: 110 - DNA, 24 bp, synthetic construct
  source 1..24: mol_type = other DNA
taccgagctcggatccgccaccat

SEQ ID NO: 111 - DNA, 27 bp, synthetic construct
  source 1..27: mol_type = other DNA
gggccctctagactcgagtcagtgatg

SEQ ID NO: 112 - DNA, 36 bp, synthetic construct
  source 1..36: mol_type = other DNA
ggtgttacaatatttcacgttgatagacttggctac

SEQ ID NO: 113 - DNA, 36 bp, synthetic construct
  source 1..36: mol_type = other DNA
gtagccaagtctatcaacgtgaaatattgtaacacc

SEQ ID NO: 114 - DNA, 36 bp, synthetic construct
  source 1..36: mol_type = other DNA
tctggaattgtaatacacggaaccctcagtataatg

SEQ ID NO: 115 - DNA, 36 bp, synthetic construct
  source 1..36: mol_type = other DNA
cattatactgagggttccgtgtattacaattccaga

SEQ ID NO: 116 - DNA, 36 bp, synthetic construct
  source 1..36: mol_type = other DNA
ggaattgtaatatggggaagactcagtataatgcga

SEQ ID NO: 117 - DNA, 36 bp, synthetic construct
  source 1..36: mol_type = other DNA
tcgcattatactgagtcttccccatattacaattcc

SEQ ID NO: 118 - DNA, 37 bp, synthetic construct
  source 1..37: mol_type = other DNA
ggaattgtaatatggggaaccaagagtataatgcgaa

SEQ ID NO: 119 - DNA, 37 bp, synthetic construct
  source 1..37: mol_type = other DNA
ttcgcattatactcttggttccccatattacaattcc

SEQ ID NO: 120 - DNA, 33 bp, synthetic construct
  source 1..33: mol_type = other DNA
attgtaatatggggagccctcagtataatgcga

SEQ ID NO: 121 - DNA, 33 bp, synthetic construct
  source 1..33: mol_type = other DNA
tcgcattatactgagggctccccatattacaat

SEQ ID NO: 122 - DNA, 33 bp, synthetic construct
  source 1..33: mol_type = other DNA
gtgaatggaggaatctgccagaagatttcctta

SEQ ID NO: 123 - DNA, 32 bp, synthetic construct
  source 1..32: mol_type = other DNA
aaggaaatcttctggcagattcctccattcac

SEQ ID NO: 124 - DNA, 33 bp, synthetic construct
  source 1..33: mol_type = other DNA
caagaatttgaaaagtgcgggaaggatttcctg

SEQ ID NO: 125 - DNA, 33 bp, synthetic construct
  source 1..33: mol_type = other DNA
caggaaatccttcccgcacttttcaaattcttg

SEQ ID NO: 126 - DNA, 34 bp, synthetic construct
  source 1..34: mol_type = other DNA
cttgtgtatacattttgcgaaactgttgctctgg

SEQ ID NO: 127 - DNA, 34 bp, synthetic construct
  source 1..34: mol_type = other DNA
ccagagcaacagtttcgcaaaatgtatacacaag

SEQ ID NO: 128 - DNA, 29 bp, synthetic construct
  source 1..29: mol_type = other DNA
tggaattcaagtgactgccttcacctcaa

SEQ ID NO: 129 - DNA, 30 bp, synthetic construct
  source 1..30: mol_type = other DNA
gaaggcagtcacttgaattccagtttttcc

SEQ ID NO: 130 - DNA, 30 bp, synthetic construct
  source 1..30: mol_type = other DNA
cttcggccaaatcactgctggggttattat

SEQ ID NO: 131 - DNA, 30 bp, synthetic construct
  source 1..30: mol_type = other DNA
ataataaccccagcagtgatttggccgaag

SEQ ID NO: 132 - DNA, 33 bp, synthetic construct
  source 1..33: mol_type = other DNA
tggaatgaaagcacttgcctttacccatccatt

SEQ ID NO: 133 - DNA, 29 bp, synthetic construct
  source 1..29: mol_type = other DNA
ggatgggtaaaggcaagtgctttcattcc

SEQ ID NO: 134 - DNA, 35 bp, synthetic construct
  source 1..35: mol_type = other DNA
ctgaatttcagagcatgccctgttattccaaatgt

SEQ ID NO: 135 - DNA, 35 bp, synthetic construct
  source 1..35: mol_type = other DNA
acatttggaataacagggcatgctctgaaattcag

SEQ ID NO: 136 - DNA, 33 bp, synthetic construct
  source 1..33: mol_type = other DNA
ttttattgcagctgttgcagcaccttgagttgt

SEQ ID NO: 137 - DNA, 33 bp, synthetic construct
  source 1..33: mol_type = other DNA
acaactcaaggtgctgcaacagctgcaataaaa

SEQ ID NO: 138 - DNA, 33 bp, synthetic construct
  source 1..33: mol_type = other DNA
gagccactagatatgtgcctcttctctttcata

SEQ ID NO: 139 - DNA, 32 bp, synthetic construct
  source 1..32: mol_type = other DNA
atgaaagagaagaggcacatatctagtggctc

SEQ ID NO: 140 - DNA, 33 bp, synthetic construct
  source 1..33: mol_type = other DNA
gatgtgtgtattgcttgcggtgtctgtatagat

SEQ ID NO: 141 - DNA, 33 bp, synthetic construct
  source 1..33: mol_type = other DNA
atctatacagacaccgcaagcaatacacacatc

(19) United States
(12) Patent Application Publication
(10) Pub. No.: US 2026/0366004 A1
(43) Pub. Date: Nov. 19, 2026

(54) NOVEL HYALURONIDASE PH-20 VARIANT, AND USE THEREOF

(71) Applicant: PNP BIOPHARM CO., LTD., Seoul (KR)

(72) Inventors: Hang Cheol SHIN, Seoul (KR); Joo Young LEE, Seoul (KR)

(21) Appl. No.: 19/112,429

(22) PCT Filed: Aug. 24, 2023
(86) PCT No.: PCT/KR2023/095052

§ 371 Date: Jan. 23, 2026

(30) Foreign Application Priority Data
Sep. 16, 2022 (KR) 10-2022-0117475

(51) Int. Cl. C12N 9/26; A61K 38/00; A61K 39/395; A61K 45/06

(52) U.S. Cl. CPC C12N 9/2474; A61K 39/3955; A61K 45/06; C12Y 302/01035; A61K 38/00

(57) ABSTRACT

The present invention relates to a novel hyaluronidase PH-20 variant, and a use thereof, and more specifically, to: a hyaluronidase PH-20 variant selected from the group consisting of SEQ ID NO: 2, SEQ ID NO: 3, and SEQ ID NO: 4; a polynucleotide encoding the variant; an expression vector comprising the polynucleotide; a host cell line including the expression vector; a method for producing a hyaluronidase PH-20 variant, the method comprising culturing the host cell line; and a pharmaceutical composition for preventing or treating diseases, the composition comprising the hyaluronidase PH-20 variant.

### TECHNICAL FIELD

The present application claims priority to Korean Patent Application No. 10-2022-0117475, filed on Sep. 16, 2022, and the entire disclosure of the specification is incorporated herein by reference.

The present invention relates to novel hyaluronidase PH-20 variants and uses thereof, and more specifically, to a hyaluronidase PH-20 variant selected from the group consisting of SEQ ID NO: 2, SEQ ID NO: 3, and SEQ ID NO: 4, a polynucleotide encoding the variant, an expression vector comprising the polypeptide, a host cell line comprising the expression vector, a method for producing the hyaluronidase PH-20 variant comprising the step of culturing the host cell line, and a pharmaceutical composition for preventing or treating a disease comprising the hyaluronidase PH-20 variant.

### BACKGROUND OF THE INVENTION

Hyaluronidase is an enzyme that degrades hyaluronic acid. In humans, there are five types of hyaluronidases: Hyal1, Hyal2, Hyal3, Hyal4, and Hyal5 (also known as PH-20 or SPAM1), and in addition, there is a pseudogene, Hyal6P (Hyalp1), which is known not to be expressed. The Hyal1, Hyal2, and Hyal3 genes are located on chromosome 3, while the Hyal4, Hyal5, and Hyal6P genes are located on chromosome 7. In humans, Hyal1 and Hyal2 are hyaluronidases expressed in most tissues and exhibit optimal activity at pH 3-4. In contrast, PH-20 is expressed on the sperm cell membrane and acrosomal membrane and has activity over a broad pH range of 3-8.

PH-20 was first identified in guinea pig sperm by Lathrop et al., and it has been reported to be expressed in the sperm of many other species. The human PH-20 gene is encoded by the SPAM1 (sperm adhesion molecule 1) gene, and PH-20 exists on the sperm cell membrane surface and the inner side of the acrosomal membrane in a form where Ser490 of PH-20 is bound to glycosylphosphatidylinositol (GPI). Sperm PH-20 plays a role in hydrolyzing hyaluronic acid when penetrating the cumulus layer of the egg, which is rich in hyaluronic acid, to enter the egg.

Additionally, due to its specificity of having activity even at neutral pH, PH-20 is also administered together with therapeutic drugs. That is, drugs requiring high doses or large amounts, particularly antibody drugs, are generally administered via intravenous injection. In this case, the injection time alone takes about 90 minutes or more, and additional preparation work for intravenous injection is required, causing inconvenience to both patients and medical staff and incurring additional costs. In contrast, subcutaneous injection has the advantage of allowing immediate administration, but compared to intravenous injection, it has lower absorption rates and slower absorption. Therefore, when the injection volume exceeds 3-5 mL, it can cause swelling and pain at the injection site. For this reason, subcutaneous injection of protein therapeutics has typically been limited to small volumes of 2 mL or less. However, when hyaluronidase PH-20 is administered subcutaneously together with therapeutic drugs, hyaluronic acid distributed in the extracellular matrix of the skin is hydrolyzed by the action of hyaluronidase, reducing the viscosity of the subcutaneous area and increasing permeability, thereby facilitating the delivery of high-dose pharmaceuticals into the body.

Accordingly, research is being conducted to develop various therapeutic drugs into subcutaneous injection formulations using PH-20. Currently, PH-20 that is commercially used is extracted from the testes of cows or sheep. However, when animal-derived hyaluronidase is repeatedly administered in high doses to humans, neutralizing antibodies may be generated, and other animal-derived substances included as impurities besides PH-20 may pose a risk of causing allergies. To address these issues, research using human PH-20 protein is being conducted. However, since the stability of human PH-20 itself is low, it is necessary to overcome this issue first.

Therefore, there is a need to develop human-derived PH-20 variants that minimize mutations introduced into wild-type PH-20 while maximizing protein stability and hyaluronidase activity.

### Problem to be Solved

Accordingly, the present inventors have completed the present invention by developing a novel PH-20 variant that minimizes the issue of immunogenicity by reducing mutations introduced into the human-derived wild-type PH-20 protein to a minimum, and enhances the stability of the protein and maximizes enzymatic activity through covalent bonding and mutations of amino acids located inside the protein.

Therefore, an object of the present invention is to provide a hyaluronidase PH-20 variant selected from the group consisting of SEQ ID NO: 2, SEQ ID NO: 3, and SEQ ID NO: 4, and further comprising 0 to 3 mutations.

Another object of the present invention is to provide a polynucleotide encoding the hyaluronidase PH-20 variant, an expression vector comprising the polynucleotide, a host cell line comprising the expression vector, and a method for producing the hyaluronidase PH-20 variant comprising the step of culturing the host cell line.

Another object of the present invention is to provide a pharmaceutical composition for preventing or treating a disease, comprising: a) a drug; and b) the hyaluronidase PH-20 variant.

Additionally, another object of the present invention is to provide a pharmaceutical composition for preventing or treating a disease, consisting of: a) a drug; and b) the hyaluronidase PH-20 variant.

Additionally, another object of the present invention is to provide a pharmaceutical composition for preventing or treating a disease, consisting essentially of: a) a drug; and b) the hyaluronidase PH-20 variant.

Another object of the present invention is to provide the use of: a) a drug; and b) the hyaluronidase PH-20 variant; for manufacturing a pharmaceutical composition for treating a disease.

Another object of the present invention is to provide a method for treating a disease, comprising administering to a subject in need thereof an effective amount of a composition comprising as active ingredients: a) a drug; and b) the hyaluronidase PH-20 variant.

### Means for Solving the Problem

To achieve the above objectives, the present invention provides a hyaluronidase PH-20 variant selected from the group consisting of SEQ ID NO: 2, SEQ ID NO: 3, and SEQ ID NO: 4, wherein 0 to 3 additional mutations are introduced.

To achieve another objective of the present invention, the present invention provides a polynucleotide encoding the hyaluronidase PH-20 variant, an expression vector comprising the polynucleotide, a host cell line comprising the expression vector, and a method for producing the hyaluronidase PH-20 variant comprising culturing the host cell line.

To achieve another objective of the present invention, the present invention provides a pharmaceutical composition for preventing or treating a disease, comprising: a) a drug; and b) the hyaluronidase PH-20 variant.

To achieve another objective of the present invention, the present invention provides a pharmaceutical composition for preventing or treating a disease, consisting of: a) a drug; and b) the hyaluronidase PH-20 variant.

To achieve another objective of the present invention, the present invention provides a pharmaceutical composition for preventing or treating a disease, consisting essentially of: a) a drug; and b) the hyaluronidase PH-20 variant.

To achieve another objective of the present invention, the present invention provides the use of a) a drug; and b) the hyaluronidase PH-20 variant; for manufacturing a pharmaceutical composition for treating a disease.

To achieve another objective of the present invention, the present invention provides a method for treating a disease, comprising administering to a subject in need thereof an effective amount of a composition comprising as active ingredients: a) a drug; and b) the hyaluronidase PH-20 variant.

The present invention will now be described in detail.

The present invention provides a hyaluronidase PH-20 variant selected from the group consisting of SEQ ID NO: 2, SEQ ID NO: 3, and SEQ ID NO: 4.

The human wild-type PH-20 protein is a protein having the amino acid sequence of SEQ ID NO: 1, and its protein structure has not yet been elucidated. Among human hyaluronidases, the protein structure of Hyal1 has been elucidated, and the amino acid sequence of Hyal1 and PH-20 shows 35.1% identity.

Amino acid sequence of human wild-type PH-20
SEQ ID NO: 1
MGVLKFKHIF FRSFVKSSGV SQIVFTFLLI
PCCLTLNFRA PPVIPNVPFL WAWNAPSEFC
LGKFDEPLDM SLFSFIGSPR INATGQGVTI
FYVDRLGYYP YIDSITGVTV NGGIPQKISL
QDHLDKAKKD ITFYMPVDNL GMAVIDWEEW
RPTWARNWKP KDVYKNRSIE LVQQQNVQLS
LTEATEKAKQ EFEKAGKDFL VETIKLGKLL
RPNHLWGYYL FPDCYNHHYK KPGYNGSCFN
VEIKRNDDLS WLWNESTALY PSIYLNTQQS
PVAATLYVRN RVREAIRVSK IPDAKSPLPV
FAYTRIVFTD QVLKFLSQDE LVYTFGETVA
LGASGIVIWG TLSIMRSMKS CLLLDNYMET
ILNPYIINVT LAAKMCSQVL CQEQGVCIRK
NWNSSDYLHL NPDNFAIQLE KGGKFTVRGK
PTLEDLEQFS EKFYCSCYST LSCKEKADVK
DTDAVDVCIA DGVCIDAFLK PPMETEEPQI
FYNASPSTLS ATMFIVSILF LIISSVASL.

The inventors of the present invention constructed the Hy1 protein of SEQ ID NO: 2, in which the signal peptide (1st to 35th amino acid residues) and part of the C-terminal sequence of the human wild-type PH-20 amino acid sequence were removed. Considering that modifications at the N-terminal and C-terminal could affect enzymatic activity, the inventors further constructed Hy2 (SEQ ID NO: 3), in which asparagine was added to the C-terminal of the Hy1 amino acid sequence, and Hy3 (SEQ ID NO: 4), in which two amino acid residues, leucine and asparagine, were deleted from the N-terminal of the Hy2 amino acid sequence.

In the present invention, the amino acid residue positions of each variant are based on the amino acid positions of Hy1 according to SEQ ID NO: 2.

Based on the amino acid sequence of SEQ ID NO: 2, additional mutations were introduced through processes of active site modeling and disulfide bond modeling.

For active site modeling, protein tertiary structure modeling was performed using AlphaFold2 by DeepMind and the CSA (conformational space annealing) modeling technique by Good Intelligence Co., Ltd. Local energy minimization was performed using the GROMACS package, and the CHARMM36 force field was applied. To create various variants from the PH-20 modeled structure, Chimera and PyMOL software were used. Additionally, various prediction programs were used to predict the protein-ligand binding affinity of each variant.

For disulfide bond modeling, candidate residues capable of forming disulfide bonds were selected using the structures of proteins registered in the PDB. Using a protein contact map visualization program, residues were analyzed by plotting those with a C-alpha carbon distance of 6 Å or less and a C-beta carbon distance of 5 Å. Subsequently, the formation of disulfide bonds was analyzed using the YASARA Web Energy Minimization Server, and energy minimization was performed using the AMBER force field FF99 in Chimera. Mutations were selected such that the RMSD measurement value of the generated protein structure aligned with the wild-type PH-20 was 0.5 or less.

Accordingly, in one embodiment of the present invention, a hyaluronidase PH-20 variant is provided, which is selected from the group consisting of SEQ ID NO: 2, SEQ ID NO: 3, and SEQ ID NO: 4, and further comprises a substitution of any amino acid residue selected from the group consisting of i) Y57H, ii) W304H, iii) T306R, vi) L307K, and v) T306A.

In another embodiment of the present invention, a hyaluronidase PH-20 variant is provided, which is selected from the group consisting of SEQ ID NO: 2, SEQ ID NO: 3, and SEQ ID NO: 4, and further comprises a substitution of any amino acid residue selected from the group consisting of i) Y57H, ii) W304H, iii) T306R, and vi) L307K.

In another embodiment of the present invention, a hyaluronidase PH-20 variant is provided, which is selected from the group consisting of SEQ ID NO: 2, SEQ ID NO: 3, and SEQ ID NO: 4, and further comprises a substitution of any amino acid residue selected from the group consisting of i) P80C and A160C, ii) G291C and Y362C, iii) L180C and A223C, vi) P6C and Y403C, and v) S36C and D426C.

In another embodiment of the present invention, a hyaluronidase PH-20 variant is provided, which is selected from the group consisting of SEQ ID NO: 2, SEQ ID NO: 3, and SEQ ID NO: 4, and further comprises a substitution of any amino acid residue selected from the group consisting of i) G291C and Y362C, ii) L180C and A223C, iii) P6C and Y403C, and vi) S36C and D426C.

In another embodiment of the present invention, a hyaluronidase PH-20 variant is provided, which is selected from the group consisting of SEQ ID NO: 2, SEQ ID NO: 3, and SEQ ID NO: 4, or further comprises a substitution of any amino acid residue selected from the group consisting of i) Y57H, ii) W304H, iii) T306R, vi) L307K, and v) T306A, and further comprises a substitution of any amino acid residue selected from the group consisting of i) P80C and A160C, ii) G291C and Y362C, iii) L180C and A223C, vi) P6C and Y403C, and v) S36C and D426C.

In the present invention, expressions such as “Y57” refer to the amino acid residue at each position in the amino acid sequence according to SEQ ID NO: 2. For example, “Y57” means that the amino acid residue at the 57th position in the amino acid sequence of SEQ ID NO: 2 is tyrosine.

Also, “Y57H” means that the 57th tyrosine of SEQ ID NO: 2 is substituted with histidine.

The PH-20 variant according to the present invention is interpreted to include variants in which amino acid residues are conservatively substituted at specific amino acid residue positions.

The term ‘conservative substitution’ refers to a modification of the PH-20 variant that includes substituting one or more amino acids with amino acids having similar biochemical properties that do not cause a loss of the biological or biochemical function of the PH-20 variant. ‘Conservative amino acid substitution’ is a substitution in which an amino acid residue is replaced with an amino acid residue having a similar side chain. Classes of amino acid residues with similar side chains are well known in the art. These classes comprise amino acids with basic side chains (e.g., lysine, arginine, histidine), amino acids with acidic side chains (e.g., aspartic acid, glutamic acid), amino acids with uncharged polar side chains (e.g., glycine, asparagine, glutamine, serine, threonine, tyrosine, cysteine), amino acids with non-polar side chains (e.g., alanine, valine, leucine, isoleucine, proline, phenylalanine, methionine, tryptophan), amino acids with beta-branched side chains (e.g., threonine, valine, isoleucine), and amino acids with aromatic side chains (e.g., tyrosine, phenylalanine, tryptophan, histidine).

In the present invention, it is expected that the PH-20 variant may still retain activity even if it has conservative amino acid substitutions. Furthermore, the PH-20 variant according to the present invention is interpreted to include PH-20 variants that have substantially the same function and/or effect as the PH-20 variant of the present invention and have an amino acid sequence homology of 80% or 85% or more, preferably 90% or more, more preferably 95% or more, and most preferably 9900 or more.

Preferably, the hyaluronidase PH-20 variant according to the present invention may be any one selected from the group consisting of amino acid sequences of SEQ ID NOs: 2 to 109. The amino acid sequences of the hyaluronidase PH-20 variants produced in the examples of the present invention are described in Table 1 below.

TABLE 1

| SEQ | | | |
|---|---|---|---|
| ID | | | |
| NO. | Name | Variant | Amino Acid Sequence |
| 2 | Hy1 | | LNFRAPPVIPNVPFLWAWNAPSEFCLGKFDEPLDMSLFSFIGSPRINATGQG |
| | | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 3 | Hy2 | | LNFRAPPVIPNVPFLWAWNAPSEFCLGKFDEPLDMSLFSFIGSPRINATGQG |
| | | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 4 | Hy3 | | FRAPPVIPNVPFLWAWNAPSEFCLGKFDEPLDMSLFSFIGSPRINATGQGVTI |
| | | | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 5 | Hy1- | P80C, | LNFRAPPVIPNVPFLWAWNAPSEFCLGKFDEPLDMSLFSFIGSPRINATGQG |
| | M1 | A160C | VTIFYVDRLGYYPYIDSITGVTVNGGICQKISLQDHLDKAKKDITFYMPVD |
| | | | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 6 | Hy1- | G291C, | LNFRAPPVIPNVPFLWAWNAPSEFCLGKFDEPLDMSLFSFIGSPRINATGQG |
| | M2 | Y362C | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWGT |
| | | | LSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 7 | Hy1- | L180C, | LNFRAPPVIPNVPFLWAWNAPSEFCLGKFDEPLDMSLFSFIGSPRINATGQG |
| | M3 | A223C | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 8 | Hy1- | P6C, | LNFRACPVIPNVPFLWAWNAPSEFCLGKFDEPLDMSLFSFIGSPRINATGQG |
| | M4 | Y403C | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFY |
| 9 | Hy1- | S36C, | LNFRAPPVIPNVPFLWAWNAPSEFCLGKFDEPLDMCLFSFIGSPRINATGQG |
| | M5 | D426C | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFY |
| 10 | Hy1- | Y57H | LNFRAPPVIPNVPFLWAWNAPSEFCLGKFDEPLDMSLFSFIGSPRINATGQG |
| | A1 | | VTIFHVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 11 | Hy1- | Y57H, | LNFRAPPVIPNVPFLWAWNAPSEFCLGKFDEPLDMSLFSFIGSPRINATGQG |
| | A1-M1 | P80C, | VTIFHVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 12 | Hy1- | Y57H, | LNFRAPPVIPNVPFLWAWNAPSEFCLGKFDEPLDMSLFSFIGSPRINATGQG |
| | A1-M2 | G291C, | VTIFHVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWGT |
| | | | LSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 13 | Hy1- | Y57H, | LNFRAPPVIPNVPFLWAWNAPSEFCLGKFDEPLDMSLFSFIGSPRINATGQG |
| | A1-M3 | L180C, | VTIFHVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 14 | Hy1- | Y57H, | LNFRACPVIPNVPFLWAWNAPSEFCLGKFDEPLDMSLFSFIGSPRINATGQG |
| | A1-M4 | P6C, | VTIFHVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFY |
| 15 | Hy1- | Y57H, | LNFRAPPVIPNVPFLWAWNAPSEFCLGKFDEPLDMCLFSFIGSPRINATGQG |
| | A1-M5 | S36C, | VTIFHVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFY |
| 16 | Hy1- | W304H | LNFRAPPVIPNVPFLWAWNAPSEFCLGKFDEPLDMSLFSFIGSPRINATGQG |
| | A2 | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 17 | Hy1- | W304H, | LNFRAPPVIPNVPFLWAWNAPSEFCLGKFDEPLDMSLFSFIGSPRINATGQG |
| | A2-M1 | P80C, | VTIFYVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 18 | Hy1- | W304H, | LNFRAPPVIPNVPFLWAWNAPSEFCLGKFDEPLDMSLFSFIGSPRINATGQG |
| | A2-M2 | G291C, | VTIFYVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIHGT |
| | | | LSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 19 | Hy1- | W304H, | LNFRAPPVIPNVPFLWAWNAPSEFCLGKFDEPLDMSLFSFIGSPRINATGQG |
| | A2-M3 | L180C, | VTIFYVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 20 | Hy1- | W304H, | LNFRACPVIPNVPFLWAWNAPSEFCLGKFDEPLDMSLFSFIGSPRINATGQG |
| | A2-M4 | P6C, | VTIFYVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFY |
| 21 | Hy1- | W304H, | LNFRAPPVIPNVPFLWAWNAPSEFCLGKFDEPLDMCLFSFIGSPRINATGQG |
| | A2-M5 | S36C, | VTIFHVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFY |
| 22 | Hy1- | T306R | LNFRAPPVIPNVPFLWAWNAPSEFCLGKFDEPLDMSLFSFIGSPRINATGQG |
| | A3 | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 23 | Hy1- | T306R, | LNFRAPPVIPNVPFLWAWNAPSEFCLGKFDEPLDMSLFSFIGSPRINATGQG |
| | A3-M1 | P80C, | VTIFYVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 24 | Hy1- | T306R, | LNFRAPPVIPNVPFLWAWNAPSEFCLGKFDEPLDMSLFSFIGSPRINATGQG |
| | A3-M2 | G291C, | VTIFYVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWG |
| | | | RLSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 25 | Hy1- | T306R, | LNFRAPPVIPNVPFLWAWNAPSEFCLGKFDEPLDMSLFSFIGSPRINATGQG |
| | A3-M3 | L180C, | VTIFYVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 26 | Hy1- | T306R, | LNFRACPVIPNVPFLWAWNAPSEFCLGKFDEPLDMSLFSFIGSPRINATGQG |
| | A3-M4 | P6C, | VTIFYVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFY |
| 27 | Hy1- | T306R, | LNFRAPPVIPNVPFLWAWNAPSEFCLGKFDEPLDMCLFSFIGSPRINATGQG |
| | A3-M5 | S36C, | VTIFYVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFY |
| 28 | Hy1- | L307K | LNFRAPPVIPNVPFLWAWNAPSEFCLGKFDEPLDMSLFSFIGSPRINATGQG |
| | A4 | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 29 | Hy1- | L307K, | LNFRAPPVIPNVPFLWAWNAPSEFCLGKFDEPLDMSLFSFIGSPRINATGQG |
| | A4-M1 | P80C, | VTIFYVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 30 | Hy1- | L307K, | LNFRAPPVIPNVPFLWAWNAPSEFCLGKFDEPLDMSLFSFIGSPRINATGQG |
| | A4-M2 | G291C, | VTIFYVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWGT |
| | | | KSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 31 | Hy1- | L307K, | LNFRAPPVIPNVPFLWAWNAPSEFCLGKFDEPLDMSLFSFIGSPRINATGQG |
| | A4-M3 | L180C, | VTIFYVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 32 | Hy1- | L307K, | LNFRACPVIPNVPFLWAWNAPSEFCLGKFDEPLDMSLFSFIGSPRINATGQG |
| | A4-M4 | P6C, | VTIFYVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFY |
| 33 | Hy1- | L307K, | LNFRAPPVIPNVPFLWAWNAPSEFCLGKFDEPLDMCLFSFIGSPRINATGQG |
| | A4-M5 | S36C, | VTIFYVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFY |
| 34 | Hy1- | T306A | LNFRAPPVIPNVPFLWAWNAPSEFCLGKFDEPLDMSLFSFIGSPRINATGQG |
| | A5 | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 35 | Hy1- | T306A, | LNFRAPPVIPNVPFLWAWNAPSEFCLGKFDEPLDMSLFSFIGSPRINATGQG |
| | A5-M1 | P80C, | VTIFYVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 36 | Hy1- | T306A, | LNFRAPPVIPNVPFLWAWNAPSEFCLGKFDEPLDMSLFSFIGSPRINATGQG |
| | A5-M2 | G291C, | VTIFYVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWG |
| | | | ALSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 37 | Hy1- | T306A, | LNFRAPPVIPNVPFLWAWNAPSEFCLGKFDEPLDMSLFSFIGSPRINATGQG |
| | A5-M3 | L180C, | VTIFYVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFY |
| 38 | Hy1- | T306A, | LNFRACPVIPNVPFLWAWNAPSEFCLGKFDEPLDMSLFSFIGSPRINATGQG |
| | A5-M4 | P6C, | VTIFYVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFY |
| 39 | Hy1- | T306A, | LNFRAPPVIPNVPFLWAWNAPSEFCLGKFDEPLDMCLFSFIGSPRINATGQG |
| | A5-M5 | S36C, | VTIFYVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFY |
| 40 | Hy2- | P80C, | LNFRAPPVIPNVPFLWAWNAPSEFCLGKFDEPLDMSLFSFIGSPRINATGQG |
| | M1 | A160C | VTIFYVDRLGYYPYIDSITGVTVNGGICQKISLQDHLDKAKKDITFYMPVD |
| | | | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 41 | Hy2- | G291C, | LNFRAPPVIPNVPFLWAWNAPSEFCLGKFDEPLDMSLFSFIGSPRINATGQG |
| | M2 | Y362C | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWGT |
| | | | LSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 42 | Hy2- | L180C, | LNFRAPPVIPNVPFLWAWNAPSEFCLGKFDEPLDMSLFSFIGSPRINATGQG |
| | M3 | A223C | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 43 | Hy2- | P6C, | LNFRACPVIPNVPFLWAWNAPSEFCLGKFDEPLDMSLFSFIGSPRINATGQG |
| | M4 | Y403C | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFYN |
| 44 | Hy2- | S36C, | LNFRAPPVIPNVPFLWAWNAPSEFCLGKFDEPLDMCLFSFIGSPRINATGQG |
| | M5 | D426C | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFYN |
| 45 | Hy2- | Y57H | LNFRAPPVIPNVPFLWAWNAPSEFCLGKFDEPLDMSLFSFIGSPRINATGQG |
| | A1 | | VTIFHVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 46 | Hy2- | Y57H, | LNFRAPPVIPNVPFLWAWNAPSEFCLGKFDEPLDMSLFSFIGSPRINATGQG |
| | A1-M1 | P80C, | VTIFHVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 47 | Hy2- | Y57H, | LNFRAPPVIPNVPFLWAWNAPSEFCLGKFDEPLDMSLFSFIGSPRINATGQG |
| | A1-M2 | G291C, | VTIFHVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWGT |
| | | | LSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 48 | Hy2- | Y57H, | LNFRAPPVIPNVPFLWAWNAPSEFCLGKFDEPLDMSLFSFIGSPRINATGQG |
| | A1-M3 | L180C, | VTIFHVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 49 | Hy2- | Y57H, | LNFRACPVIPNVPFLWAWNAPSEFCLGKFDEPLDMSLFSFIGSPRINATGQG |
| | A1-M4 | P6C, | VTIFHVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFYN |
| 50 | Hy2- | Y57H, | LNFRAPPVIPNVPFLWAWNAPSEFCLGKFDEPLDMCLFSFIGSPRINATGQG |
| | A1-M5 | S36C, | VTIFHVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFYN |
| 51 | Hy2- | W304H | LNFRAPPVIPNVPFLWAWNAPSEFCLGKFDEPLDMSLFSFIGSPRINATGQG |
| | A2 | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 52 | Hy2- | W304H, | LNFRAPPVIPNVPFLWAWNAPSEFCLGKFDEPLDMSLFSFIGSPRINATGQG |
| | A2-M1 | P80C, | VTIFYVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 53 | Hy2- | W304H, | LNFRAPPVIPNVPFLWAWNAPSEFCLGKFDEPLDMSLFSFIGSPRINATGQG |
| | A2-M2 | G291C, | VTIFYVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIHGT |
| | | | LSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 54 | Hy2- | W304H, | LNFRAPPVIPNVPFLWAWNAPSEFCLGKFDEPLDMSLFSFIGSPRINATGQG |
| | A2-M3 | L180C, | VTIFYVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 55 | Hy2- | W304H, | LNFRACPVIPNVPFLWAWNAPSEFCLGKFDEPLDMSLFSFIGSPRINATGQG |
| | A2-M4 | P6C, | VTIFYVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFYN |
| 56 | Hy2- | W304H, | LNFRAPPVIPNVPFLWAWNAPSEFCLGKFDEPLDMCLFSFIGSPRINATGQG |
| | A2-M5 | S36C, | VTIFHVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIHGT |
| | | | LSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFYN |
| 57 | Hy2- | T306R | LNFRAPPVIPNVPFLWAWNAPSEFCLGKFDEPLDMSLFSFIGSPRINATGQG |
| | A3 | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 58 | Hy2- | T306R, | LNFRAPPVIPNVPFLWAWNAPSEFCLGKFDEPLDMSLFSFIGSPRINATGQG |
| | A3-M1 | P80C, | VTIFYVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 59 | Hy2- | T306R, | LNFRAPPVIPNVPFLWAWNAPSEFCLGKFDEPLDMSLFSFIGSPRINATGQG |
| | A3-M2 | G291C, | VTIFYVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWG |
| | | | RLSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 60 | Hy2- | T306R, | LNFRAPPVIPNVPFLWAWNAPSEFCLGKFDEPLDMSLFSFIGSPRINATGQG |
| | A3-M3 | L180C, | VTIFYVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 61 | Hy2- | T306R, | LNFRACPVIPNVPFLWAWNAPSEFCLGKFDEPLDMSLFSFIGSPRINATGQG |
| | A3-M4 | P6C, | VTIFYVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFYN |
| 62 | Hy2- | T306R, | LNFRAPPVIPNVPFLWAWNAPSEFCLGKFDEPLDMCLFSFIGSPRINATGQG |
| | A3-M5 | S36C, | VTIFYVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | RLSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFYN |
| 63 | Hy2- | L307K | LNFRAPPVIPNVPFLWAWNAPSEFCLGKFDEPLDMSLFSFIGSPRINATGQG |
| | A4 | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 64 | Hy2- | L307K, | LNFRAPPVIPNVPFLWAWNAPSEFCLGKFDEPLDMSLFSFIGSPRINATGQG |
| | A4-M1 | P80C. | VTIFYVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 65 | Hy2- | L307K, | LNFRAPPVIPNVPFLWAWNAPSEFCLGKFDEPLDMSLFSFIGSPRINATGQG |
| | A4-M2 | G291C, | VTIFYVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWGT |
| | | | KSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 66 | Hy2- | L307K, | LNFRAPPVIPNVPFLWAWNAPSEFCLGKFDEPLDMSLFSFIGSPRINATGQG |
| | A4-M3 | L180C, | VTIFYVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 67 | Hy2- | L307K, | LNFRACPVIPNVPFLWAWNAPSEFCLGKFDEPLDMSLFSFIGSPRINATGQG |
| | A4-M4 | P6C, | VTIFYVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFYN |
| 68 | Hy2- | L307K, | LNFRAPPVIPNVPFLWAWNAPSEFCLGKFDEPLDMCLFSFIGSPRINATGQG |
| | A4-M5 | S36C, | VTIFYVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | TKSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFYN |
| 69 | Hy2- | T306A | LNFRAPPVIPNVPFLWAWNAPSEFCLGKFDEPLDMSLFSFIGSPRINATGQG |
| | A5 | | VTIFYVDRLGYYPYIDSITGVTVNGGIPQKISLQDHLDKAKKDITFYMPVD |
| | | | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 70 | Hy2- | T306A, | LNFRAPPVIPNVPFLWAWNAPSEFCLGKFDEPLDMSLFSFIGSPRINATGQG |
| | A5-M1 | P80C, | VTIFYVDRLGYYPYIDSITGVTVNGGICQKISLQDHLDKAKKDITFYMPVD |
| | | A160C | NLGMAVIDWEEWRPTWARNWKPKDVYKNRSIELVQQQNVQLSLTEATEK |
| | | | AKQEFEKCGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 71 | Hy2- | T306A, | LNFRAPPVIPNVPFLWAWNAPSEFCLGKFDEPLDMSLFSFIGSPRINATGQG |
| | A5-M2 | G291C, | VTIFYVDRLGYYPYIDSITGVTVNGGIPQKISLQDHLDKAKKDITFYMPVD |
| | | Y362C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFCETVALGASGIVIWG |
| | | | ALSIMRSMKSCLLLDNYMETILNPYIINVTLAAKMCSQVLCQEQGVCIRKN |
| | | | WNSSDCLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 72 | Hy2- | T306A, | LNFRAPPVIPNVPFLWAWNAPSEFCLGKFDEPLDMSLFSFIGSPRINATGQG |
| | A5-M3 | L180C, | VTIFYVDRLGYYPYIDSITGVTVNGGIPQKISLQDHLDKAKKDITFYMPVD |
| | | A223C | NLGMAVIDWEEWRPTWARNWKPKDVYKNRSIELVQQQNVQLSLTEATEK |
| | | | AKQEFEKAGKDFLVETIKLGKLLRPNHCWGYYLFPDCYNHHYKKPGYNG |
| | | | SCFNVEIKRNDDLSWLWNESTCLYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIADGVCIDAFLKPPMETEEPQIFYN |
| 73 | Hy2- | T306A, | LNFRACPVIPNVPFLWAWNAPSEFCLGKFDEPLDMSLFSFIGSPRINATGQG |
| | A5-M4 | P6C, | VTIFYVDRLGYYPYIDSITGVTVNGGIPQKISLQDHLDKAKKDITFYMPVD |
| | | Y403C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCCSTLS |
| | | | CKEKADVKDTDAVDVCIADGVCIDAFLKPPMETEEPQIFYN |
| 74 | Hy2- | T306A, | LNFRAPPVIPNVPFLWAWNAPSEFCLGKFDEPLDMCLFSFIGSPRINATGQG |
| | A5-M5 | S36C, | VTIFYVDRLGYYPYIDSITGVTVNGGIPQKISLQDHLDKAKKDITFYMPVD |
| | | D426C | NLGMAVIDWEEWRPTWARNWKPKDVYKNRSIELVQQQNVQLSLTEATEK |
| | | | AKQEFEKAGKDFLVETIKLGKLLRPNHLWGYYLFPDCYNHHYKKPGYNG |
| | | | SCFNVEIKRNDDLSWLWNESTALYPSIYLNTQQSPVAATLYVRNRVREAIRV |
| | | | SKIPDAKSPLPVFAYTRIVFTDQVLKFLSQDELVYTFGETVALGASGIVIWG |
| | | | ALSIMRSMKSCLLLDNYMETILNPYIINVTLAAKMCSQVLCQEQGVCIRKN |
| | | | WNSSDYLHLNPDNFAIQLEKGGKFTVRGKPTLEDLEQFSEKFYCSCYSTLS |
| | | | CKEKADVKDTDAVDVCIACGVCIDAFLKPPMETEEPQIFYN |
| 75 | Hy3- | P80C, | FRAPPVIPNVPFLWAWNAPSEFCLGKFDEPLDMSLFSFIGSPRINATGQGVTI |
| | M1 | A160C | FYVDRLGYYPYIDSITGVTVNGGICQKISLQDHLDKAKKDITFYMPVDNLG |
| | | | MAVIDWEEWRPTWARNWKPKDVYKNRSIELVQQQNVQLSLTEATEKAKQ |
| | | | EFEKCGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 76 | Hy3- | G291C, | FRAPPVIPNVPFLWAWNAPSEFCLGKFDEPLDMSLFSFIGSPRINATGQGVTI |
| | M2 | Y362C | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFCETVALGASGIVIWGTLSI |
| | | | MRSMKSCLLLDNYMETILNPYIINVTLAAKMCSQVLCQEQGVCIRKNWNS |
| | | | SDCLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 77 | Hy3- | L180C, | FRAPPVIPNVPFLWAWNAPSEFCLGKFDEPLDMSLFSFIGSPRINATGQGVTI |
| | M3 | A223C | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHCWGYYLFPDCYNHHYKKPGYNGSCF |
| | | | NVEIKRNDDLSWLWNESTCLYPSIYLNTQQSPVAATLYVRNRVREAIRVSKI |
| | | | PDAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 78 | Hy3- | P6C, | FRACPVIPNVPFLWAWNAPSEFCLGKFDEPLDMSLFSFIGSPRINATGQGVTI |
| | M4 | Y403C | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCCSTLSCKE |
| | | | KADVKDTDAVDVCIADGVCIDAFLKPPMETEEPQIFYN |
| 79 | Hy3- | S36C, | FRAPPVIPNVPFLWAWNAPSEFCLGKFDEPLDMCLFSFIGSPRINATGQGVTI |
| | M5 | D426C | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIACGVCIDAFLKPPMETEEPQIFYN |
| 80 | Hy3- | Y57H | FRAPPVIPNVPFLWAWNAPSEFCLGKFDEPLDMSLFSFIGSPRINATGQGVTI |
| | A1 | | FHVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 81 | Hy3- | Y57H, | FRAPPVIPNVPFLWAWNAPSEFCLGKFDEPLDMSLFSFIGSPRINATGQGVTI |
| | A1-M1 | P80C, | FHVDRLGYYPYIDSITGVTVNGGICQKISLQDHLDKAKKDITFYMPVDNLG |
| | | A160C | MAVIDWEEWRPTWARNWKPKDVYKNRSIELVQQQNVQLSLTEATEKAKQ |
| | | | EFEKCGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 82 | Hy3- | Y57H, | FRAPPVIPNVPFLWAWNAPSEFCLGKFDEPLDMSLFSFIGSPRINATGQGVTI |
| | A1-M2 | G291C, | FHVDRLGYYPYIDSITGVTVNGGIPQKISLQDHLDKAKKDITFYMPVDNLG |
| | | Y362C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFCETVALGASGIVIWGTLSI |
| | | | MRSMKSCLLLDNYMETILNPYIINVTLAAKMCSQVLCQEQGVCIRKNWNS |
| | | | SDCLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 83 | Hy3- | Y57H, | FRAPPVIPNVPFLWAWNAPSEFCLGKFDEPLDMSLFSFIGSPRINATGQGVTI |
| | A1-M3 | L180C, | FHVDRLGYYPYIDSITGVTVNGGIPQKISLQDHLDKAKKDITFYMPVDNLG |
| | | A223C | MAVIDWEEWRPTWARNWKPKDVYKNRSIELVQQQNVQLSLTEATEKAKQ |
| | | | EFEKAGKDFLVETIKLGKLLRPNHCWGYYLFPDCYNHHYKKPGYNGSCF |
| | | | NVEIKRNDDLSWLWNESTCLYPSIYLNTQQSPVAATLYVRNRVREAIRVSKI |
| | | | PDAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 84 | Hy3- | Y57H, | FRACPVIPNVPFLWAWNAPSEFCLGKFDEPLDMSLFSFIGSPRINATGQGVTI |
| | A1-M4 | P6C, | FHVDRLGYYPYIDSITGVTVNGGIPQKISLQDHLDKAKKDITFYMPVDNLG |
| | | Y403C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCCSTLSCKE |
| | | | KADVKDTDAVDVCIADGVCIDAFLKPPMETEEPQIFYN |
| 85 | Hy3- | Y57H, | FRAPPVIPNVPFLWAWNAPSEFCLGKFDEPLDMCLFSFIGSPRINATGQGVTI |
| | A1-M5 | S36C, | FHVDRLGYYPYIDSITGVTVNGGIPQKISLQDHLDKAKKDITFYMPVDNLG |
| | | D426C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIACGVCIDAFLKPPMETEEPQIFYN |
| 86 | Hy3- | W304H | FRAPPVIPNVPFLWAWNAPSEFCLGKFDEPLDMSLFSFIGSPRINATGQGVTI |
| | A2 | | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIHGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 87 | Hy3- | W304H, | FRAPPVIPNVPFLWAWNAPSEFCLGKFDEPLDMSLFSFIGSPRINATGQGVTI |
| | A2-M1 | P80C, | FYVDRLGYYPYIDSITGVTVNGGICQKISLQDHLDKAKKDITFYMPVDNLG |
| | | A160C | MAVIDWEEWRPTWARNWKPKDVYKNRSIELVQQQNVQLSLTEATEKAKQ |
| | | | EFEKCGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIHGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 88 | Hy3- | W304H, | FRAPPVIPNVPFLWAWNAPSEFCLGKFDEPLDMSLFSFIGSPRINATGQGVTI |
| | A2-M2 | G291C, | FYVDRLGYYPYIDSITGVTVNGGIPQKISLQDHLDKAKKDITFYMPVDNLG |
| | | Y362C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFCETVALGASGIVIHGTLSIM |
| | | | RSMKSCLLLDNYMETILNPYIINVTLAAKMCSQVLCQEQGVCIRKNWNSS |
| | | | DCLHLNPDNFAIQLEKGGKFTVRGKPTLEDLEQFSEKFYCSCYSTLSCKEK |
| | | | ADVKDTDAVDVCIADGVCIDAFLKPPMETEEPQIFYN |
| 89 | Hy3- | W304H, | FRAPPVIPNVPFLWAWNAPSEFCLGKFDEPLDMSLFSFIGSPRINATGQGVTI |
| | A2-M3 | L180C, | FYVDRLGYYPYIDSITGVTVNGGIPQKISLQDHLDKAKKDITFYMPVDNLG |
| | | A223C | MAVIDWEEWRPTWARNWKPKDVYKNRSIELVQQQNVQLSLTEATEKAKQ |
| | | | EFEKAGKDFLVETIKLGKLLRPNHCWGYYLFPDCYNHHYKKPGYNGSCF |
| | | | NVEIKRNDDLSWLWNESTCLYPSIYLNTQQSPVAATLYVRNRVREAIRVSKI |
| | | | PDAKSPLPVFAYTRIVFTDQVLKFLSQDELVYTFGETVALGASGIVIHGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 90 | Hy3- | W304H, | FRACPVIPNVPFLWAWNAPSEFCLGKFDEPLDMSLFSFIGSPRINATGQGVTI |
| | A2-M4 | P6C, | FYVDRLGYYPYIDSITGVTVNGGIPQKISLQDHLDKAKKDITFYMPVDNLG |
| | | Y403C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIHGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCCSTLSCKE |
| | | | KADVKDTDAVDVCIADGVCIDAFLKPPMETEEPQIFYN |
| 91 | Hy3- | W304H, | FRAPPVIPNVPFLWAWNAPSEFCLGKFDEPLDMCLFSFIGSPRINATGQGVTI |
| | A2-M5 | S36C, | FHVDRLGYYPYIDSITGVTVNGGIPQKISLQDHLDKAKKDITFYMPVDNLG |
| | | D426C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIHGTLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIACGVCIDAFLKPPMETEEPQIFYN |
| 92 | Hy3- | T306R | FRAPPVIPNVPFLWAWNAPSEFCLGKFDEPLDMSLFSFIGSPRINATGQGVTI |
| | A3 | | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGRLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 93 | Hy3- | T306R, | FRAPPVIPNVPFLWAWNAPSEFCLGKFDEPLDMSLFSFIGSPRINATGQGVTI |
| | A3-M1 | P80C, | FYVDRLGYYPYIDSITGVTVNGGICQKISLQDHLDKAKKDITFYMPVDNLG |
| | | A160C | MAVIDWEEWRPTWARNWKPKDVYKNRSIELVQQQNVQLSLTEATEKAKQ |
| | | | EFEKCGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGRLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 94 | Hy3- | T306R, | FRAPPVIPNVPFLWAWNAPSEFCLGKFDEPLDMSLFSFIGSPRINATGQGVTI |
| | A3-M2 | G291C, | FYVDRLGYYPYIDSITGVTVNGGIPQKISLQDHLDKAKKDITFYMPVDNLG |
| | | Y362C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFCETVALGASGIVIWGRLSI |
| | | | MRSMKSCLLLDNYMETILNPYIINVTLAAKMCSQVLCQEQGVCIRKNWNS |
| | | | SDCLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 95 | Hy3- | T306R, | FRAPPVIPNVPFLWAWNAPSEFCLGKFDEPLDMSLFSFIGSPRINATGQGVTI |
| | A3-M3 | L180C, | FYVDRLGYYPYIDSITGVTVNGGIPQKISLQDHLDKAKKDITFYMPVDNLG |
| | | A223C | MAVIDWEEWRPTWARNWKPKDVYKNRSIELVQQQNVQLSLTEATEKAKQ |
| | | | EFEKAGKDFLVETIKLGKLLRPNHCWGYYLFPDCYNHHYKKPGYNGSCF |
| | | | NVEIKRNDDLSWLWNESTCLYPSIYLNTQQSPVAATLYVRNRVREAIRVSKI |
| | | | PDAKSPLPVFAYTRIVFTDQVLKFLSQDELVYTFGETVALGASGIVIWGRLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 96 | Hy3- | T306R, | FRACPVIPNVPFLWAWNAPSEFCLGKFDEPLDMSLFSFIGSPRINATGQGVTI |
| | A3-M4 | P6C, | FYVDRLGYYPYIDSITGVTVNGGIPQKISLQDHLDKAKKDITFYMPVDNLG |
| | | Y403C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGRLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCCSTLSCKE |
| | | | KADVKDTDAVDVCIADGVCIDAFLKPPMETEEPQIFYN |
| 97 | Hy3- | T306R, | FRAPPVIPNVPFLWAWNAPSEFCLGKFDEPLDMCLFSFIGSPRINATGQGVTI |
| | A3-M5 | S36C, | FYVDRLGYYPYIDSITGVTVNGGIPQKISLQDHLDKAKKDITFYMPVDNLG |
| | | D426C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGRLSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIACGVCIDAFLKPPMETEEPQIFYN |
| 98 | Hy3- | L307K | FRAPPVIPNVPFLWAWNAPSEFCLGKFDEPLDMSLFSFIGSPRINATGQGVTI |
| | A4 | | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTKSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 99 | Hy3- | L307K, | FRAPPVIPNVPFLWAWNAPSEFCLGKFDEPLDMSLFSFIGSPRINATGQGVTI |
| | A4-M1 | P80C, | FYVDRLGYYPYIDSITGVTVNGGICQKISLQDHLDKAKKDITFYMPVDNLG |
| | | A160C | MAVIDWEEWRPTWARNWKPKDVYKNRSIELVQQQNVQLSLTEATEKAKQ |
| | | | EFEKCGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTKSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 100 | Hy3- | L307K, | FRAPPVIPNVPFLWAWNAPSEFCLGKFDEPLDMSLFSFIGSPRINATGQGVTI |
| | A4-M2 | G291C, | FYVDRLGYYPYIDSITGVTVNGGIPQKISLQDHLDKAKKDITFYMPVDNLG |
| | | Y362C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFCETVALGASGIVIWGTKSI |
| | | | MRSMKSCLLLDNYMETILNPYIINVTLAAKMCSQVLCQEQGVCIRKNWNS |
| | | | SDCLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 101 | Hy3- | L307K, | FRAPPVIPNVPFLWAWNAPSEFCLGKFDEPLDMSLFSFIGSPRINATGQGVTI |
| | A4-M3 | L180C, | FYVDRLGYYPYIDSITGVTVNGGIPQKISLQDHLDKAKKDITFYMPVDNLG |
| | | A223C | MAVIDWEEWRPTWARNWKPKDVYKNRSIELVQQQNVQLSLTEATEKAKQ |
| | | | EFEKAGKDFLVETIKLGKLLRPNHCWGYYLFPDCYNHHYKKPGYNGSCF |
| | | | NVEIKRNDDLSWLWNESTCLYPSIYLNTQQSPVAATLYVRNRVREAIRVSKI |
| | | | PDAKSPLPVFAYTRIVFTDQVLKFLSQDELVYTFGETVALGASGIVIWGTKS |
| | | | IMRSMKSCLLLDNYMETILNPYIINVTLAAKMCSQVLCQEQGVCIRKNWN |
| | | | SSDYLHLNPDNFAIQLEKGGKFTVRGKPTLEDLEQFSEKFYCSCYSTLSCK |
| | | | EKADVKDTDAVDVCIADGVCIDAFLKPPMETEEPQIFYN |
| 102 | Hy3- | L307K, | FRACPVIPNVPFLWAWNAPSEFCLGKFDEPLDMSLFSFIGSPRINATGQGVTI |
| | A4-M4 | P6C, | FYVDRLGYYPYIDSITGVTVNGGIPQKISLQDHLDKAKKDITFYMPVDNLG |
| | | Y403C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTKSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCCSTLSCKE |
| | | | KADVKDTDAVDVCIADGVCIDAFLKPPMETEEPQIFYN |
| 103 | Hy3- | L307K, | FRAPPVIPNVPFLWAWNAPSEFCLGKFDEPLDMCLFSFIGSPRINATGQGVTI |
| | A4-M5 | S36C, | FYVDRLGYYPYIDSITGVTVNGGIPQKISLQDHLDKAKKDITFYMPVDNLG |
| | | D426C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGTKSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIACGVCIDAFLKPPMETEEPQIFYN |
| 104 | Hy3- | T306A | FRAPPVIPNVPFLWAWNAPSEFCLGKFDEPLDMSLFSFIGSPRINATGQGVTI |
| | A5 | | FYVDRLGYYPYIDSITGVTVNGGIPQKISLQDHLDKAKKDITFYMPVDNLG |
| | | | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGALSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 105 | Hy3- | T306A, | FRAPPVIPNVPFLWAWNAPSEFCLGKFDEPLDMSLFSFIGSPRINATGQGVTI |
| | A5-M1 | P80C, | FYVDRLGYYPYIDSITGVTVNGGICQKISLQDHLDKAKKDITFYMPVDNLG |
| | | A160C | MAVIDWEEWRPTWARNWKPKDVYKNRSIELVQQQNVQLSLTEATEKAKQ |
| | | | EFEKCGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGALSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 106 | Hy3- | T306A, | FRAPPVIPNVPFLWAWNAPSEFCLGKFDEPLDMSLFSFIGSPRINATGQGVTI |
| | A5-M2 | G291C, | FYVDRLGYYPYIDSITGVTVNGGIPQKISLQDHLDKAKKDITFYMPVDNLG |
| | | Y362C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFCETVALGASGIVIWGALSI |
| | | | MRSMKSCLLLDNYMETILNPYIINVTLAAKMCSQVLCQEQGVCIRKNWNS |
| | | | SDCLHLNPDNFAIQLEKGGKFTVRGKPTLEDLEQFSEKFYCSCYSTLSCKE |
| | | | KADVKDTDAVDVCIADGVCIDAFLKPPMETEEPQIFYN |
| 107 | Hy3- | T306A, | FRAPPVIPNVPFLWAWNAPSEFCLGKFDEPLDMSLFSFIGSPRINATGQGVTI |
| | A5-M3 | L180C, | FYVDRLGYYPYIDSITGVTVNGGIPQKISLQDHLDKAKKDITFYMPVDNLG |
| | | A223C | MAVIDWEEWRPTWARNWKPKDVYKNRSIELVQQQNVQLSLTEATEKAKQ |
| | | | EFEKAGKDFLVETIKLGKLLRPNHCWGYYLFPDCYNHHYKKPGYNGSCF |
| | | | NVEIKRNDDLSWLWNESTCLYPSIYLNTQQSPVAATLYVRNRVREAIRVSKI |
| | | | PDAKSPLPVFAYTRIVFTDQVLKFLSQDELVYTFGETVALGASGIVIWGALS |
| | | | IMRSMKSCLLLDNYMETILNPYIINVTLAAKMCSQVLCQEQGVCIRKNWN |
| | | | SSDYLHLNPDNFAIQLEKGGKFTVRGKPTLEDLEQFSEKFYCSCYSTLSCK |
| | | | EKADVKDTDAVDVCIADGVCIDAFLKPPMETEEPQIFYN |
| 108 | Hy3- | T306A, | FRACPVIPNVPFLWAWNAPSEFCLGKFDEPLDMSLFSFIGSPRINATGQGVTI |
| | A5-M4 | P6C, | FYVDRLGYYPYIDSITGVTVNGGIPQKISLQDHLDKAKKDITFYMPVDNLG |
| | | Y403C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGALSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCCSTLSCKE |
| | | | KADVKDTDAVDVCIADGVCIDAFLKPPMETEEPQIFYN |
| 109 | Hy3- | T306A, | FRAPPVIPNVPFLWAWNAPSEFCLGKFDEPLDMCLFSFIGSPRINATGQGVTI |
| | A5-M5 | S36C, | FYVDRLGYYPYIDSITGVTVNGGIPQKISLQDHLDKAKKDITFYMPVDNLG |
| | | D426C | MAVIDWEEWRPTWARNWKPKDVYKNRSIELVQQQNVQLSLTEATEKAKQ |
| | | | EFEKAGKDFLVETIKLGKLLRPNHLWGYYLFPDCYNHHYKKPGYNGSCFN |
| | | | VEIKRNDDLSWLWNESTALYPSIYLNTQQSPVAATLYVRNRVREAIRVSKIP |
| | | | DAKSPLPVFAYTRIVFTDQVLKFLSQDELVYTFGETVALGASGIVIWGALSI |
| | | | MRSMKSCLLLDNYMETILNPYIINVTLAAKMCSQVLCQEQGVCIRKNWNS |
| | | | SDYLHLNPDNFAIQLEKGGKFTVRGKPTLEDLEQFSEKFYCSCYSTLSCKE |
| | | | KADVKDTDAVDVCIACGVCIDAFLKPPMETEEPQIFYN |

Additionally, the present invention provides a polynucleotide encoding the hyaluronidase PH-20 variant according to the present invention.

The polynucleotide of the present invention may exist in cells, in cell lysates, in a partially purified form, or in a substantially pure form. The polynucleotide is ‘isolated’ or ‘substantially pure’ when it is purified from other cellular components or other contaminants, such as polynucleotides or proteins of other cells, by standard techniques including, but not limited to, alkali/SDS treatment, CsCl banding, column chromatography, agarose gel electrophoresis, and other methods well known in the art. The polynucleotide is not particularly limited as long as it is a combination of sequences capable of encoding the PH-20 variant of the present invention. The polynucleotide may be provided as a nucleic acid molecule in the form of a single-stranded or double-stranded DNA, cDNA, or RNA sequence.

The nucleic acid sequence encoding the PH-20 variant of the present invention can be operably linked to a vector capable of expressing it to provide the PH-20 variant of the present invention.

Accordingly, the present invention provides an expression vector or recombinant vector comprising the polynucleotide.

In the present invention, the term ‘expression vector’ or ‘recombinant vector’ refers to a genetic construct comprising essential regulatory elements operably linked to allow the expression of a target protein or target RNA in an appropriate host cell.

The term ‘operably linked’ refers to a functional linkage between a nucleic acid expression regulatory sequence and a nucleic acid sequence encoding a desired protein or RNA so that they can perform their general functions. For example, a promoter and a nucleic acid sequence encoding a protein or RNA are operably linked to influence the expression of the coding nucleic acid sequence. Operable linkage with a recombinant vector can be prepared using gene recombination techniques well known in the art, and site-specific DNA cleavage and ligation are generally performed using enzymes well known in the art.

In one embodiment, the vector may include, but is not limited to, plasmid vectors, cosmid vectors, bacteriophage vectors, and viral vectors. Suitable expression vectors may include expression regulatory elements such as promoters, operators, initiation codons, termination codons, polyadenylation signals, and enhancers, as well as signal sequences or leader sequences for membrane targeting or secretion, and may be variously constructed depending on the purpose. The promoter of the vector may be constitutive or inducible. Additionally, the expression vector may include a selection marker for selecting host cells containing the vector and, in the case of a replicable expression vector, may include an origin of replication.

Signal sequences may include, for example, PhoA signal sequences or OmpA signal sequences when the host is a bacterium of the genus Escherichia, u-amylase signal sequences or subtilisin signal sequences when the host is a bacterium of the genus Bacillus, MFu signal sequences or SUC2 signal sequences when the host is yeast, and insulin signal sequences, α-interferon signal sequences, or antibody molecule signal sequences when the host is an animal cell, but are not limited thereto.

Additionally, the present invention provides a host cell line comprising the expression vector. That is, the present invention provides a transformant (host cell) transformed with the expression vector (recombinant vector).

The transformation may be performed by any method known to introduce nucleic acids into organisms, cells, tissues, or organs, and may be carried out by selecting appropriate standard techniques depending on the host cell, as known in the art. Such methods include, but are not limited to, microprojectile bombardment, electroporation, protoplast fusion, calcium phosphate (CaPO₄) precipitation, calcium chloride (CaCl₂)) precipitation, agitation using silicon carbide fibers, Agrobacterium-mediated transformation, PEG-mediated fusion, microinjection, liposome-mediated methods, dextran sulfate, lipofectamine, and heat shock methods.

The term ‘transformant’ can be used interchangeably with ‘host cell’ and refers to a prokaryotic or eukaryotic cell containing heterologous DNA introduced into the cell by any means (e.g., electroporation, calcium phosphate precipitation, microinjection, transformation, viral infection, etc.).

In the present invention, the transformant may use as a host cell any type of unicellular organism commonly used in the field of cloning, such as prokaryotic microorganisms including various bacteria (e.g., Clostridia, Escherichia coli, etc.), lower eukaryotic microorganisms such as yeast, and cells derived from higher eukaryotic organisms including insect cells, plant cells, and mammalian cells, but is not limited thereto. Since the expression level and modification of the protein may vary depending on the host cell, a person skilled in the art can select and use the host cell most suitable for the intended purpose. The transformant of the present invention may preferably mean a transformed microorganism. Specifically, but not limited to, the host cell may be a prokaryotic host cell such as Escherichia coli, Bacillus subtilis, Streptomyces, Pseudomonas, Proteus mirabilis, or Staphylococcus. Additionally, lower eukaryotic cells, such as fungi (e.g., Aspergillus), yeast (e.g., Pichia pastoris, Saccharomyces cerevisiae, Schizosaccharomyces, Neurospora crassa), cells derived from higher eukaryotic organisms comprising insect cells, plant cells, and mammalian cells, may be used as host cells. Preferably, monkey kidney cells 7 (COS7), NSO cells, SP2/0, Chinese hamster ovary (CHO) cells, W138, baby hamster kidney (BHK) cells, MDCK, myeloma cell lines, HuT 78 cells, and HEK293 cells may be used, and particularly preferably, CHO cells may be used, but the invention is not limited thereto.

The present invention also provides a method for producing a hyaluronidase PH-20 variant, comprising the step of culturing the host cell line.

When a recombinant expression vector capable of expressing the PH-20 variant or a fragment thereof is introduced into a host cell, the PH-20 variant can be produced by culturing the host cell for a sufficient period of time to allow the PH-20 variant to be expressed in the host cell, and more preferably, for a sufficient period of time to allow the PH-20 variant to be secreted into the culture medium in which the host cell is cultured.

The composition of the medium, culture conditions, and culture time for culturing the host cell can be appropriately selected according to methods commonly used in the art. The PH-20 variant produced in the host cell may accumulate in the cytoplasm of the cell, be secreted outside the cell or into the culture medium by an appropriate signal sequence, or be targeted to the periplasm, etc.

In addition, it is preferable to refold the protein using methods known in the art so that the PH-20 variant according to the present invention maintains its enzymatic activity and has a functional conformation.

In some cases, the expressed PH-20 variant may be isolated from the host cell and purified to homogeneity. The isolation or purification of the PH-20 variant can be performed by separation and purification methods commonly used for proteins, for example, chromatography. The chromatography may be one or more combinations selected from, for example, affinity chromatography, ion-exchange chromatography, or hydrophobic chromatography, but is not limited thereto. In addition to chromatography, filtration, ultrafiltration, salting out, dialysis, etc., may be used in combination.

The present invention also provides a pharmaceutical composition for preventing or treating a disease, comprising: a) a drug; and b) the hyaluronidase PH-20 variant.

In the pharmaceutical composition according to the present invention, the drug may include protein drugs, antibody drugs, small molecules, aptamers, RNAi, antisense, CAR-T cells (chimeric antigen receptor T cells), or CAR-NK cells (CAR natural killer cells), but is not limited thereto, and both drugs currently commercialized and in use, as well as drugs under clinical and developmental stages, can be used. Preferably, protein drugs or antibody drugs may be used as the drug.

In the pharmaceutical composition according to the present invention, the “protein drug” refers to a drug composed of amino acids that exhibits disease prevention or treatment effects through the activity of the protein, and means a pharmaceutical product composed of proteins other than antibody drugs. It may be selected from the group consisting of cytokines, therapeutic enzymes, hormones, soluble receptors and their fusion proteins, insulin or its analogs, BMP (bone morphogenetic protein), EPO (erythropoietin), and serum-derived proteins, but is not limited thereto.

The “cytokine” may be selected from the group consisting of interferons, interleukins, CSF (colony stimulating factor), TNF (tumor necrosis factor), and TGF (tissue growth factor), but is not limited thereto, and the “therapeutic enzyme” may include, for example, β-glucocerebrosidase and agalsidase β, but is not limited thereto.

The “soluble receptor” refers to the extracellular domain of a receptor, and its fusion protein refers to a protein in which the Fc region of an antibody or the like is fused to the soluble receptor. The soluble receptor is a soluble form of a receptor to which a ligand associated with a disease binds, and examples include a form in which the Fc region is fused to a TNF-α soluble receptor (e.g., a product with the generic name etanercept and similar forms), a form in which the Fc region is fused to a VEGF soluble receptor (e.g., a product with the generic name aflibercept and similar forms), a form in which the Fc region is fused to CTLA-4 (e.g., a product with the generic name abatacept or belatacept and similar forms), a form in which the Fc region is fused to an interleukin 1 soluble receptor (e.g., a product with the generic name rilonacept and similar forms), and a form in which the Fc region is fused to an LFA3 soluble receptor (e.g., a product with the generic name alefacept and similar forms), but is not limited thereto.

The “hormone” refers to a hormone or its analog administered exogenously for the treatment or prevention of diseases caused by hormone deficiency, and examples include human growth hormone, estrogen, and progesterone, but is not limited thereto. The “serum-derived protein” refers to proteins present in serum, including those extracted from serum and those produced recombinantly, and examples include fibrinogen, von Willebrand Factor, albumin, thrombin, Factor II, Factor V, Factor VII, Factor VIII, Factor IX, Factor X, and Factor XI, but is not limited thereto.

In the pharmaceutical composition according to the present invention, the “antibody drug” may be a monoclonal antibody drug or a polyclonal antibody drug.

The ‘monoclonal antibody drug’ refers to a protein comprising monoclonal antibodies and monoclonal antibody fragments that can specifically bind to antigens associated with certain diseases. Monoclonal antibodies also include bispecific antibodies, and proteins comprising monoclonal antibodies or fragments thereof are used in the sense of including ADCs (antibody-drug conjugates). Antigens associated with certain diseases include, but are not limited to, 4-1BB, integrin, amyloid beta, angiopoietin (angiopoietin 1 or 2), angiopoietin-like 3, B-cell activating factor (BAFF), B7-H3, complement 5, CCR4, CD3, CD4, CD6, CD11a, CD19, CD20, CD22, CD30, CD33, CD38, CD52, CD62, CD79b, CD80, CGRP, Claudin-18, complement factor D, CTLA4, DLL3, EGF receptor, hemophilia factor, Fc receptor, FGF23, folate receptor, GD2, GM-CSF, HER2, HER3, interferon receptor, interferon gamma, IgE, IGF-1 receptor, interleukin 1, interleukin 2 receptor, interleukin 4 receptor, interleukin 5, interleukin 5 receptor, interleukin 6, interleukin 6 receptor, interleukin 7, interleukin 12/23, interleukin 13, interleukin 17A, interleukin 17 receptor A, interleukin 31 receptor, interleukin 36 receptor, LAG3, LFA3, NGF, PVSK9, PD-1, PD-L1, RANK-L, SLAMF7, tissue factor, TNF, VEGF, VEGF receptor, and vWF (von Willebrand Factor). The following are proteins comprising monoclonal antibodies and monoclonal antibody fragments against the antigens associated with certain diseases:
An antibody against 4-1BB: Utomilumab;
Antibodies against integrin: Natalizumab, Etrolizumab, Vedolizumab, Bimagrumab;
Antibodies against amyloid beta: Bapineuzumab, Crenezumab, Solanezumab, Aducanumab, Gantenerumab;
Antibodies against angiopoietin: AMG 780 for angiopoietin 1 and 2, MEDI 3617 for angiopoietin 2, Nesvacumab, and Vanucizumab, a bispecific antibody against angiopoietin 2 and VEGF;
An antibody against angiopoietin-like 3: Evinacumab;
Antibodies against B-cell activating factor (BAFF): Tabalumab, Lanalumab, Belimumab;
An antibody against B7-H3: Omburtamab;
Antibodies against complement 5: Ravulizumab, Eculizumab;
An antibody against CCR4: Mogamulizumab;
Antibodies against CD3: Otelixizumab, Teplizumab, Muromonab, Tebentafusp (a bispecific antibody against GP100 and CD3), Blinatumomab (a bispecific antibody against CD19 and CD3), REGN1979 (a bispecific antibody against CD20 and CD3);
Antibodies against CD4: Ibalizumab, Zanolimumab;
An antibody against CD6: Itolizumab;
An antibody against CD11a: Efalizumab;
Antibodies against CD19: Inebilizumab, Tafasitamab, and Loncastuximab tesirine (an ADC);
Antibodies against CD20: Ocrelizumab, Ublituximab, Obinutuzumab, Ofatumumab, Rituximab, Tositumomab, and Ibritumomab tiuxetan (an ADC);
Antibodies against CD22: Epratuzumab, Inotuzumab ozogamicin (an ADC), Moxetumomab pasudotox (an ADC);
An ADC against CD30: Brentuximab vedotin;
ADCs against CD33: Vadastuximab talirine, Gemtuzumab ozogamicin;
Antibodies against CD38: Daratumumab, Isatuximab;
An antibody against CD52: Alemtuzumab;
An antibody against CD62: Crizanlizumab;
An ADC against CD79b: Polatuzumab vedotin;
An antibody against CD80: Galiximab;
Antibodies against CGRP: Eptinezumab, Fremanezumab, Galcanezumab, Erenumab;
An antibody against Claudin-18: Zolbetuximab;
An antibody against complement factor D: Lampalizumab;
Antibodies against CTLA4: Tremelimumab, Zalifrelimab, Ipilimumab;
An ADC against DLL3: Rovalpituzumab tesirine;
Antibodies against EGF receptor: Cetuximab, Depatuxizumab, Zalutumumab, Necitumumab, Panitumumab;
A bispecific antibody against coagulation factor IX and Factor X: Emicizumab;
Antibodies against Fc receptor: Nipocalimab, Rozanolixizumab;
An antibody against FGF23: Burosumab;
Antibodies against folate receptor: Farletuzumab, Mirvetuximab soravtansine (an ADC);
Antibodies against GD2: Dinutuximab, Naxitamab;
An antibody against GM-CSF: Otilimab;
Antibodies against HER2: Margetuximab, Pertuzumab, Trastuzumab, and ADCs of Trastuzumab deruxtecan, Trastuzumab emtansine and Trastuzumab duocarmazine;
An antibody against HER3: Patritumab;
An antibody against interferon receptor: Anifrolumab;
An antibody against interferon gamma: Emapalumab;
Antibodies against IgE: Ligelizumab, Omalizumab;
Antibodies against IGF-1 receptor: Dalotuzumab, Figitumumab, Teprotumumab;
Antibodies against interleukin 1: Gebokizumab, Canakinumab;
Antibodies against interleukin 2 receptor: Daclizumab, Basiliximab;
An antibody against interleukin 4 receptor: Dupilumab;
Antibodies against interleukin 5: Mepolizumab, Reslizumab;
An antibody against interleukin 5 receptor: Benralizumab;
Antibodies against interleukin 6: Clazakizumab, Olokizumab, Sirukumab, Siltuximab;
Antibodies against interleukin 6 receptor: Sarilumab, Satralizumab, Tocilizumab, REGN88;
An antibody against interleukin 7: Secukinumab;
Antibodies against interleukin 12/23: Ustekinumab, Briakinumab;
Antibodies against interleukin 13: Lebrikizumab, Tralokinumab;
An antibody against interleukin 17A: Ixekizumab, Bimekizumab;
An antibody against interleukin 17 receptor A: Brodalumab;
Antibodies against interleukin 23: Brazikumab, Guselkumab, Risankizumab, Tildrakizumab, Mirikizumab;
An antibody against interleukin 31 receptor: Nemolizumab;
An antibody against interleukin 36 receptor: Spesolimab;
An antibody against LAG3: Relatlimab;
An antibody against NASP2: Narsoplimab;
Antibodies against NGF: Fasinumab, Tanezumab;
Antibodies against PVSK9: Alirocumab, Evolocumab, Bococizumab;
Antibodies against PD-1: Lambrolizumab, Balstilimab, Camrelizumab, Cemiplimab, Dostarlimab, Prolgolimab, Sintilimab, Spartalizumab, Tislelizumab, Pembrolizumab, Nivolumab;
Antibodies against PD-L1: Atezolizumab, Avelumab, Envafolimab, Durvalumab, and Bintrafusp alpha (a bispecific antibody against TGF beta and PD-L1);
An antibody against RANK-L: Denosumab;
An antibody against SLAMF7: Elotuzumab;
Antibodies against tissue factor: Concizumab, Marstacimab;
Antibodies against TNF, particularly TNFα: Infliximab, Adalimumab, Golimumab, Certolizumab pegol (an antibody fragment), and Ozoralizumab (a bispecific antibody against TNF and albumin);
Antibodies against VEGF: Brolucizumab, Ranibizumab, Bevacizumab, and Faricimab (a bispecific antibody against VEGF and Ang2);
An antibody against VEGF receptor: Ramucirumab; and
An antibody against vWF: Caplacizumab.

The ‘polyclonal antibody drug’ preferably includes serum antibodies extracted from plasma, such as immune globulin, but is not limited thereto.

In the pharmaceutical composition according to the present invention, the content of the antibody drug may be 5 to 500 mg/mL, preferably 20 to 200 mg/mL, more preferably 100 to 150 mg/mL, and most preferably 120±18 mg/mL, for example, about 110 mg/mL, about 120 mg/mL, or about 130 mg/mL.

In the pharmaceutical composition according to the present invention, the ‘small molecule compound’ can be used without limitation as long as it is a drug requiring rapid action for prevention or treatment purposes. For example, analgesics of the morphine series may be used. Additionally, when used as a treatment for tissue necrosis caused by anticancer agents, it can be used alone or in combination with antidote drugs of the Vinca Alkaloids or Taxane series.

Furthermore, the pharmaceutical composition according to the present invention may further comprise one or more selected from the group consisting of a buffer, stabilizer, and surfactant.

In the pharmaceutical composition according to the present invention, the ‘buffer’ can be used without limitation as long as it provides a pH of 4 to 8, preferably 5 to 7, and preferably includes one or more selected from the group consisting of malate, formate, citrate, acetate, propionate, pyridine, piperazine, cacodylate, succinate, 2-(N-morpholino)ethanesulfonic acid (MES), histidine, Tris, bis-Tris, phosphate, ethanolamine, carbonate, piperazine-N,N′-bis(2-ethanesulfonic acid) (PIPES), imidazole, BIS-Tris propane, N,N-bis(2-hydroxyethyl)-2-aminoethanesulfonic acid (BES), 3-(N-morpholino) propanesulfonic acid (MOPS), Hydroxyethyl piperazine Ethane Sulfonic acid (HEPES), pyrophosphate, and triethanolamine, and more preferably a histidine buffer, for example, L-histidine/HCl, but is not limited thereto. The concentration of the buffer may be 0.001 to 200 mM, preferably 1 to 50 mM, more preferably 5 to 40 mM, and most preferably 10 to 30 mM.

In the pharmaceutical composition according to the present invention, the ‘stabilizer’ can be used without limitation as long as it is a substance commonly used in the art for the purpose of stabilizing proteins, and preferred examples may include one or more selected from the group consisting of carbohydrates, sugars or their hydrates, sugar alcohols or their hydrates, and amino acids.

The ‘carbohydrates, sugars, or sugar alcohols’ used as the stabilizer may include one or more selected from the group consisting of trehalose or its hydrate, sucrose, saccharin, glycerol, erythritol, threitol, xylitol, arabitol, ribitol, mannitol, sorbitol, galactitol, fucitol, iditol, inositol, volemitol, isomalt, maltitol, polyglycitol, cyclodextrin, Hydroxypropyl Beta-cyclodextrin, and glucose, but are not limited thereto. The concentration of the sugars or sugar alcohols used as the stabilizer in the pharmaceutical composition according to the present invention may be 0.001 to 500 mM, preferably 100 to 300 mM, more preferably 150 to 250 mM, and most preferably 180 to 230 mM.

The ‘amino acids’ may include one or more selected from the group consisting of glutamine, glutamic acid, glycine, lysine, lysyllysine, leucine, methionine, valine, serine, selenomethionine, citrulline, arginine, asparagine, aspartic acid, ornithine, isoleucine, taurine, theanine, threonine, tryptophan, tyrosine, phenylalanine, proline, pyrrolysine, histidine, and alanine, but are not limited thereto. The concentration of the amino acids used as the stabilizer in the pharmaceutical composition according to the present invention may be 1 to 100 mM, preferably 3 to 30 mM, more preferably 5 to 25 mM, and most preferably 7 to 20 mM.

The pharmaceutical composition according to the present invention may further comprise a surfactant. The surfactant is preferably a nonionic surfactant such as polyoxyethylene-sorbitan fatty acid ester [polysorbate or tween], polyethylene-polypropylene glycol, polyoxyethylene stearate, polyoxyethylene alkyl ether, for example, polyoxyethylene monolauryl ether, alkylphenyl polyoxyethylene ether [Triton-X], polyoxyethylene-polyoxypropylene copolymer [poloxamer, pluronic], and sodium dodecyl sulfate (SDS), and more preferably polysorbate may be used. The polysorbate may be polysorbate 20 or polysorbate 80, but not limited thereto. The concentration of the nonionic surfactant in the pharmaceutical composition according to the present invention may be 0.0000001% to 0.5% (w/v), preferably 0.000001% to 0.4% (w/v), more preferably 0.00001% to 0.3% (w/v), and most preferably 0.001% to 0.2% (w/v).

The pharmaceutical composition according to the present invention can be administered by methods such as intravenous administration, subcutaneous administration, intramuscular administration, intraperitoneal administration, endothelium administration, topical administration, intranasal administration, intrapulmonary administration, and intrarectal administration, and it is preferably administered through subcutaneous administration, and more preferably used in the form of an injection formulation for subcutaneous injection. Accordingly, in one aspect of the present invention, a subcutaneous administration formulation comprising the pharmaceutical composition according to the present invention is provided.

The subcutaneous administration formulation may be provided in a ready-to-inject form without additional dilution processes and may be provided in a pre-filled syringe or included in a glass ampoule or plastic container.

The diseases to be prevented or treated by the pharmaceutical composition according to the present invention are not particularly limited, and there is no limitation as long as the disease can be treated with a drug that can be used in combination with the PH-20 variant according to the present invention. Specifically, the diseases may comprise cancer, autoimmune diseases, etc., but are not limited thereto.

The ‘cancer’ or ‘carcinoma’ is not particularly limited and comprises both solid cancers and hematologic cancers. Examples of the cancer may include skin cancer such as melanoma, liver cancer, hepatocellular carcinoma, gastric cancer, breast cancer, lung cancer, ovarian cancer, bronchial cancer, nasopharyngeal cancer, laryngeal cancer, pancreatic cancer, bladder cancer, colon cancer, colorectal cancer, cervical cancer, brain cancer, prostate cancer, bone cancer, thyroid cancer, parathyroid cancer, kidney cancer, esophageal cancer, bile duct cancer, testicular cancer, rectal cancer, head and neck cancer, cervical cancer, ureteral cancer, osteosarcoma, neuroblastoma, fibrosarcoma, rhabdomyosarcoma, astrocytoma, neurocytoma, and glioma, and may preferably be selected from the group consisting of gastric cancer, colorectal cancer, breast cancer, lung cancer, and kidney cancer, but are not limited thereto.

The ‘autoimmune diseases’ may be selected from the group consisting of rheumatoid arthritis, asthma, psoriasis, multiple sclerosis, allergic rhinitis, Crohn's disease, ulcerative colitis, systemic lupus erythematosus, type I diabetes, inflammatory bowel disease (IBD), and atopic dermatitis, but are not limited thereto.

In addition, the present invention provides the use of a) a drug; and b) the hyaluronidase PH-20 variant; for manufacturing a pharmaceutical composition for treating a disease.

In addition, the present invention provides a method for treating a disease, comprising administering to a subject in need thereof an effective amount of a composition comprising as active ingredients: a) a drug; and b) the hyaluronidase PH-20 variant.

The ‘effective amount’ in the present invention refers to an amount that, when administered to a subject, exhibits an effect of improving, treating, detecting, diagnosing, or suppressing or reducing a disease that can be treated with a drug that can be used in combination with the PH-20 variant according to the present invention, and the ‘subject’ may be an animal, preferably a mammal, particularly an animal including a human, and may also include cells, tissues, organs, etc., derived from animals. The subject may be a patient in need of the effect.

The ‘treatment’ in the present invention comprehensively refers to improving symptoms caused by a disease that can be treated with a drug that can be used in combination with the PH-20 variant according to the present invention, and may include curing, substantially preventing, or improving the condition of the disease, and may include alleviating, curing, or preventing one or more symptoms or most symptoms caused by the disease, but is not limited thereto.

The term “comprising” as used herein is intended to have the same meaning as “including” or “characterized by”, and does not exclude additional components or steps not specifically mentioned in the composition or method according to the present invention. The term “consisting of” means excluding additional elements, steps, or components not specifically described. The term “consisting essentially of” means that the composition or method may include materials or steps that do not substantially affect the basic characteristics within the scope described.

### Effect of the Invention

Accordingly, the present invention provides a novel hyaluronidase PH-20 variant selected from the group consisting of SEQ ID NO: 1, SEQ ID NO: 2, and SEQ ID NO: 3, a polynucleotide encoding the variant, an expression vector comprising the polypeptide, a host cell line comprising the expression vector, a method for producing the hyaluronidase PH-20 variant comprising culturing the host cell line, and a pharmaceutical composition for preventing or treating a disease comprising the hyaluronidase PH-20 variant. The novel hyaluronidase PH-20 variant according to the present invention has higher stability and superior activity compared to the wild-type human hyaluronidase PH-20, thereby maximizing the therapeutic effect of drugs used in combination therewith.

### BRIEF DESCRIPTION OF THE DRAWINGS

FIG. 1 schematically illustrates the substitution positions of Y57H, W304H, T306R, L307K, and T306A amino acid residues in the hyaluronidase PH-20 variant Hy1 of the present invention.

FIG. 2 schematically illustrates the positions of disulfide bonds in the hyaluronidase PH-20 variant Hy1 of the present invention.

FIG. 3 shows the TM data of the hyaluronidase PH-20 variants Hy1, Hy1-M1, and Hy2 using the Protein Thermal Shift™ Dye Kit from Biosystems. Data for all variants are described in Table 4.

FIG. 4 shows the results of enzyme stability at 37 °C for hyaluronidase PH-20 variants. Data for all variants are described in Table 5.

FIG. 5 shows the results of enzyme stability at 50 °C for hyaluronidase PH-20 variants. Data for all variants are described in Table 6.

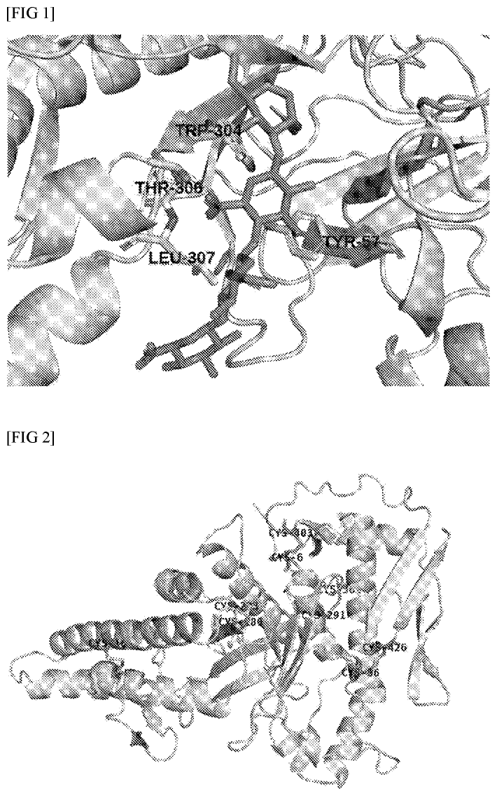

### DETAILED DESCRIPTION OF THE INVENTION

Hereinafter, the present invention will be described in detail.

However, the following examples are merely illustrative of the present invention, and the content of the present invention is not limited to the following examples.

### Example 1. 3D Modeling of Hyaluronidase PH-20 Structure

### 1-1. Active Site Modeling

For hyaluronidase PH-20, whose protein structure has not been elucidated, five initial model structures were prepared using the AlphaFold2 modeling program by DeepMind. Subsequently, the five models were used as template structures, and the final tertiary structure was completed using the protein structure modeling technique of Good Intelligence Co., Ltd.

For the amino acid residues of the active site, various variants were created through substitution with 19 different amino acids, and various prediction programs were used to predict the binding affinity of each variant to the protein and ligand. Finally, five variants predicted to have high binding affinity were selected and produced.

### 1-2. Disulfide Bond Modeling

After performing active site modeling, the process of introducing internal disulfide bonds was carried out to increase the stability of the enzyme. Disulfide bond prediction was performed using the YASARA Web server. Subsequently, to select valid variants from the proposed list, disulfide bond modeling was performed using Protein contact map visualization (Andreas Viklund), which measures distances that allow disulfide bonds.

### Example 2. Production of Hyaluronidase PH-20 Variants

Based on the results of the above modeling and simulation, hyaluronidase PH-20 variants were produced.

### 2-1. Preparation of Cloning Vector

A general mammalian cell protein expression vector and a general expression mammalian cell strain were used, and Top10 was used as E. coli strain for cloning. Gene and primer synthesis were carried out by Cosmo Genetech Co., Ltd. All restriction enzymes used for gene recombination were products of Enzynomics, and the ligase was Enzynomics' FZ-Fusion HT Cloning Kit. The DNA polymerase used for insert PCR and point mutation was PrimeSTAR HS from Takara. The DNA gel extraction kit and plasmid mini prep kit were products of Cosmo Genetech Co., Ltd. DNA sequencing was performed by Macrogen Co., Ltd.

The primers are shown in Table 2.

TABLE 2

| | primer (F) | | primer(R) | |
|---|---|---|---|---|
| Mutation | SEQ | SEQ | SEQ | |
| Name | ID | | ID | SEQ |
| Hy1, | 110 | taccgagctcggatccgcca | 111 | gggccctctagactcgagtc |
| Hy2, | | ccat | | agtgatg |
| Hy3 | | | | |
| Y57H | 112 | ggtgttacaatatttcacgt | 113 | gtagccaagtctatcaacgt |
| | | tgatagacttggctac | | gaaatattgtaacacc |
| W304H | 114 | tctggaattgtaatacacgg | 115 | cattatactgagggttccgt |
| | | aaccctcagtataatg | | gtattacaattccaga |
| T306R | 116 | ggaattgtaatatggggaag | 117 | tcgcattatactgagtcttc |
| | | actcagtataatgcga | | cccatattacaattcc |
| L307K | 118 | ggaattgtaatatggggaac | 119 | ttcgcattatactcttggtt |
| | | caagagtataatgcgaa | | ccccatattacaattcc |
| T306A | 120 | attgtaatatggggagccct | 121 | tcgcattatactgagggctc |
| | | cagtataatgcga | | cccatattacaat |
| P80C | 122 | gtgaatggaggaatctgcca | 123 | aaggaaatcttctggcagat |
| | | gaagatttcctta | | tcctccattcac |
| A160C | 124 | caagaatttgaaaagtgcgg | 125 | caggaaatccttcccgcact |
| | | gaaggatttcctg | | tttcaaattcttg |
| G291C | 126 | cttgtgtatacattttgcga | 127 | ccagagcaacagtttcgcaa |
| | | aactgttgctctgg | | aatgtatacacaag |
| Y362C | 128 | tggaattcaagtgactgcct | 129 | gaaggcagtcacttgaattc |
| | | tcacctcaa | | cagtttttcc |
| L180C | 130 | cttcggccaaatcactgctg | 131 | ataataaccccagcagtgat |
| | | gggttattat | | ttggccgaag |
| A223C | 132 | tggaatgaaagcacttgcct | 133 | ggatgggtaaaggcaagtgc |
| | | ttacccatccatt | | tttcattcc |
| P6C | 134 | ctgaatttcagagcatgccc | 135 | acatttggaataacagggca |
| | | tgttattccaaatgt | | tgctctgaaattcag |
| Y403C | 136 | ttttattgcagctgttgcag | 137 | acaactcaaggtgctgcaac |
| | | caccttgagttgt | | agctgcaataaaa |
| S36C | 138 | gagccactagatatgtgcct | 139 | atgaaagagaagaggcacat |
| | | cttctctttcata | | atctagtggctc |
| D426C | 140 | gatgtgtgtattgcttgcgg | 141 | atctatacagacaccgcaag |
| | | tgtctgtatagat | | caatacacacatc |

### 2-2. Protein Expression

Using the ExpiFectamine CHO reagent, the gene was transfected into Cricetulus griseus (CHO), and 33-38 ml of cells were cultured for 9-13 days in an orbital shaker at 37 °C, 80% humidity, and 5-8% CO₂. The expressed protein was confirmed using SDS-PAGE and western blot, filtered through a 0.22 μm filter, and used for purification.

### 2-3. Protein Purification

Purification was performed using a HisTrap HP column. Before use, the column was equilibrated with binding buffer (137 mM NaCl, 2.7 mM KCl, 10 mM Na₂HPO₄, 1.8 mM KH₂PO₄). The culture medium was passed through the column to bind the target protein to the column, and the column was washed again with binding buffer. Subsequently, to elute the target protein bound to the column, elution buffer (137 mM NaCl, 2.7 mM KCl, 10 mM Na₂HPO₄, 1.8 mM KH₂PO₄, 0.5 M imidazole) was used, and the target protein was selected using the linear gradient method. All processes were carried out at a flow rate of 3 ml/min. The target protein was filtered through a 0.2 μm filter, and its activity was measured. FPLC was performed using GE UPC-800.

### Example 3. Activity Evaluation of Hyaluronidase PH-20 Variants

The turbidimetric assay method for measuring hyaluronidase enzyme activity measures the degree of aggregate formation by the residual hyaluronic acid in the reaction solution binding to acidified albumin (BSA) through absorbance. When hyaluronic acid is hydrolyzed by hyaluronidase, the amount binding to albumin decreases, resulting in a decrease in absorbance, which is used to measure activity. Wild-type hyaluronidase was diluted to an appropriate concentration (unit/mL) using cold enzyme diluent buffer and prepared in each tube. Purified hyaluronidase samples were diluted to an appropriate concentration using cold enzyme diluent buffer and prepared in each tube. In a new tube, a 3 mg/mL hyaluronic acid solution was diluted 10-fold to a concentration of 0.3 mg/mL, and the volume of each tube was adjusted to 180 μL. To the diluted hyaluronic acid solution, 60 μL of the sample containing hyaluronidase was added and mixed (hyaluronic acid: hyaluronidase=3:1), and the reaction was carried out at 37 °C for 45 minutes. After the reaction, 50 μL of the reacted hyaluronidase and 250 μL of acidic albumin solution were added to each well of a 96-well plate, shaken at room temperature for 10 minutes, and the absorbance was measured at 600 nm using a spectrophotometer. A standard curve was created using the absorbance obtained from wild-type hyaluronidase. Using the standard curve, the activity (units/mL) of the hyaluronidase samples was calculated. The activity of each hyaluronidase was expressed as values obtained from three repetitions and their error values.

As a result, the activity of each hyaluronidase PH-20 variant was as shown in Table 3.

TABLE 3

| Seq ID NO. | Name | Variant | Exp. Act |
|---|---|---|---|
| 2 | Hy1 | | 1.000 |
| 3 | Hy2 | | 2.105 |
| 4 | Hy3 | | 3.442 |
| 5 | Hy1-M1 | P80C, A160C | 2.780 |
| 6 | Hy1-M2 | G291C, Y362C | 1.028 |
| 7 | Hy1-M3 | L180C, A223C | 2.663 |
| 8 | Hy1-M4 | P6C, Y403C | 1.001 |
| 9 | Hy1-M5 | S36C, D426C | 1.637 |
| 10 | Hy1-A1 | Y57H | 1.049 |
| 11 | Hy1-A1-M1 | Y57H, P80C, A160C | 2.916 |
| 12 | Hy1-A1-M2 | Y57H, G291C, Y362C | 1.078 |
| 13 | Hy1-A1-M3 | Y57H, L180C, A223C | 2.793 |
| 14 | Hy1-A1-M4 | Y57H, P6C, Y403C | 1.050 |
| 15 | Hy1-A1-M5 | Y57H, S36C, D426C | 1.717 |
| 16 | Hy1-A2 | W304H | 1.013 |
| 17 | Hy1-A2-M1 | W304H, P80C, A160C | 2.816 |
| 18 | Hy1-A2-M2 | W304H, G291C, Y362C | 1.042 |
| 19 | Hy1-A2-M3 | W304H, L180C, A223C | 2.698 |
| 20 | Hy1-A2-M4 | W304H, P6C, Y403C | 1.014 |
| 21 | Hy1-A2-M5 | W304H, S36C, D426C | 1.658 |
| 22 | Hy1-A3 | T306R | 1.464 |
| 23 | Hy1-A3-M1 | T306R, P80C, A160C | 4.070 |
| 24 | Hy1-A3-M2 | T306R, G291C, Y362C | 1.505 |
| 25 | Hy1-A3-M3 | T306R, L180C, A223C | 3.898 |
| 26 | Hy1-A3-M4 | T306R, P6C, Y403C | 1.465 |
| 27 | Hy1-A3-M5 | T306R, S36C, D426C | 2.396 |
| 28 | Hy1-A4 | L307K | 0.405 |
| 29 | Hy1-A4-M1 | L307K, P80C, A160C | 1.125 |
| 30 | Hy1-A4-M2 | L307K, G291C, Y362C | 0.416 |
| 31 | Hy1-A4-M3 | L307K, L180C, A223C | 1.077 |
| 32 | Hy1-A4-M4 | L307K, P6C, Y403C | 0.405 |
| 33 | Hy1-A4-M5 | L307K, S36C, D426C | 0.662 |
| 34 | Hy1-A5 | T306A | 1.144 |
| 35 | Hy1-A5-M1 | T306A, P80C, A160C | 3.180 |
| 36 | Hy1-A5-M2 | T306A, G291C, Y362C | 1.176 |
| 37 | Hy1-A5-M3 | T306A, L180C, A223C | 3.045 |
| 38 | Hy1-A5-M4 | T306A, P6C, Y403C | 1.145 |
| 39 | Hy1-A5-M5 | T306A, S36C, D426C | 1.872 |
| 40 | Hy2-M1 | P80C, A160C | 5.851 |
| 41 | Hy2-M2 | G291C, Y362C | 2.164 |
| 42 | Hy2-M3 | L180C, A223C | 5.604 |
| 43 | Hy2-M4 | P6C, Y403C | 2.107 |
| 44 | Hy2-M5 | S36C, D426C | 3.445 |
| 45 | Hy2-A1 | Y57H | 2.207 |
| 46 | Hy2-A1-M1 | Y57H, P80C, A160C | 6.137 |
| 47 | Hy2-A1-M2 | Y57H, G291C, Y362C | 2.270 |
| 48 | Hy2-A1-M3 | Y57H, L180C, A223C | 5.878 |
| 49 | Hy2-A1-M4 | Y57H, P6C, Y403C | 2.210 |
| 50 | Hy2-A1-M5 | Y57H, S36C, D426C | 3.614 |
| 51 | Hy2-A2 | W304H | 2.132 |
| 52 | Hy2-A2-M1 | W304H, P80C, A160C | 5.928 |
| 53 | Hy2-A2-M2 | W304H, G291C, Y362C | 2.192 |
| 54 | Hy2-A2-M3 | W304H, L180C, A223C | 5.677 |
| 55 | Hy2-A2-M4 | W304H, P6C, Y403C | 2.134 |
| 56 | Hy2-A2-M5 | W304H, S36C, D426C | 3.490 |
| 57 | Hy2-A3 | T306R | 3.081 |
| 58 | Hy2-A3-M1 | T306R, P80C, A160C | 8.565 |
| 59 | Hy2-A3-M2 | T306R, G291C, Y362C | 3.167 |
| 60 | Hy2-A3-M3 | T306R, L180C, A223C | 8.203 |
| 61 | Hy2-A3-M4 | T306R, P6C, Y403C | 3.084 |
| 62 | Hy2-A3-M5 | T306R, S36C, D426C | 5.043 |
| 63 | Hy2-A4 | L307K | 0.852 |
| 64 | Hy2-A4-M1 | L307K, P80C, A160C | 2.367 |
| 65 | Hy2-A4-M2 | L307K, G291C, Y362C | 0.876 |
| 66 | Hy2-A4-M3 | L307K, L180C, A223C | 2.268 |
| 67 | Hy2-A4-M4 | L307K, P6C, Y403C | 0.852 |
| 68 | Hy2-A4-M5 | L307K, S36C, D426C | 1.394 |
| 69 | Hy2-A5 | T306A | 2.407 |
| 70 | Hy2-A5-M1 | T306A, P80C, A160C | 6.692 |
| 71 | Hy2-A5-M2 | T306A, G291C, Y362C | 2.475 |
| 72 | Hy2-A5-M3 | T306A, L180C, A223C | 6.409 |
| 73 | Hy2-A5-M4 | T306A, P6C, Y403C | 2.409 |
| 74 | Hy2-A5-M5 | T306A, S36C, D426C | 3.940 |
| 75 | Hy3-M1 | P80C, A160C | 9.568 |
| 76 | Hy3-M2 | G291C, Y362C | 3.539 |
| 77 | Hy3-M3 | L180C, A223C | 9.165 |
| 78 | Hy3-M4 | P6C, Y403C | 3.445 |
| 79 | Hy3-M5 | S36C, D426C | 5.634 |
| 80 | Hy3-A1 | Y57H | 3.610 |
| 81 | Hy3-A1-M1 | Y57H, P80C, A160C | 10.036 |
| 82 | Hy3-A1-M2 | Y57H, G291C, Y362C | 3.712 |
| 83 | Hy3-A1-M3 | Y57H, L180C, A223C | 9.613 |
| 84 | Hy3-A1-M4 | Y57H, P6C, Y403C | 3.613 |
| 85 | Hy3-A1-M5 | Y57H, S36C, D426C | 5.909 |
| 86 | Hy3-A2 | W304H | 3.486 |
| 87 | Hy3-A2-M1 | W304H, P80C, A160C | 9.693 |
| 88 | Hy3-A2-M2 | W304H, G291C, Y362C | 3.585 |
| 89 | Hy3-A2-M3 | W304H, L180C, A223C | 9.284 |
| 90 | Hy3-A2-M4 | W304H, P6C, Y403C | 3.490 |
| 91 | Hy3-A2-M5 | W304H, S36C, D426C | 5.707 |
| 92 | Hy3-A3 | T306R | 5.038 |
| 93 | Hy3-A3-M1 | T306R, P80C, A160C | 14.006 |
| 94 | Hy3-A3-M2 | T306R, G291C, Y362C | 5.180 |
| 95 | Hy3-A3-M3 | T306R, L180C, A223C | 13.415 |
| 96 | Hy3-A3-M4 | T306R, P6C, Y403C | 5.043 |
| 97 | Hy3-A3-M5 | T306R, S36C, D426C | 8.247 |
| 98 | Hy3-A4 | L307K | 1.392 |
| 99 | Hy3-A4-M1 | L307K, P80C, A160C | 3.871 |
| 100 | Hy3-A4-M2 | L307K, G291C, Y362C | 1.432 |
| 101 | Hy3-A4-M3 | L307K, L180C, A223C | 3.708 |
| 102 | Hy3-A4-M4 | L307K, P6C, Y403C | 1.394 |
| 103 | Hy3-A4-M5 | L307K, S36C, D426C | 2.280 |
| 104 | Hy3-A5 | T306A | 3.936 |
| 105 | Hy3-A5-M1 | T306A, P80C, A160C | 10.943 |
| 106 | Hy3-A5-M2 | T306A, G291C, Y362C | 4.047 |
| 107 | Hy3-A5-M3 | T306A, L180C, A223C | 10.481 |
| 108 | Hy3-A5-M4 | T306A, P6C, Y403C | 3.940 |
| 109 | Hy3-A5-M5 | T306A, S36C, D426C | 6.443 |

### Example 4. Tm Analysis of Hyaluronidase PH-20 Variants

The Tmn data of hyaluronidase PH-20 variants were obtained using the Protein Thermal Shift™ Dye Kit from Biosystems. Specifically, to monitor the thermal stability of the variants, a dye binding to exposed hydrophobic residues was used, and measurements were taken with a real-time PCR device.

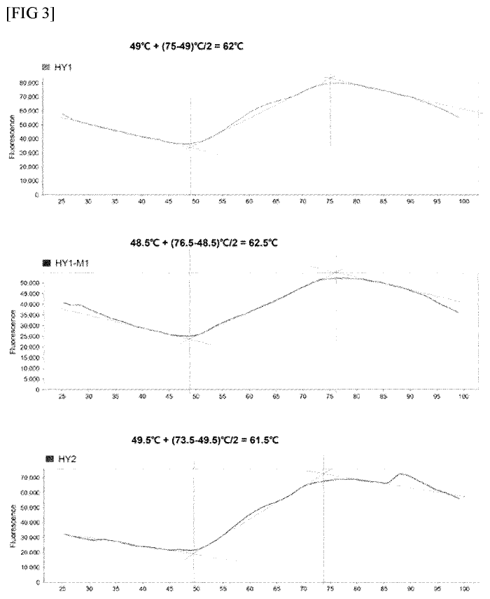

As a result, the Tm data of each hyaluronidase PH-20 variant measured were as shown in Table 4, and among them, Hy11, Hy1-M1, and Hy2 are shown in FIG. 3.

TABLE 4

| SEQ ID NO. | Name | Variant | TM |
|---|---|---|---|
| 2 | Hy1 | | 62.0 |
| 3 | Hy2 | | 61.5 |
| 4 | Hy3 | | 62.6 |
| 5 | Hy1-M1 | P80C, A160C | 62.4 |
| 6 | Hy1-M2 | G291C, Y362C | 61.5 |
| 7 | Hy1-M3 | L180C, A223C | 62.4 |
| 8 | Hy1-M4 | P6C, Y403C | 61.5 |
| 9 | Hy1-M5 | S36C, D426C | 61.9 |
| 10 | Hy1-A1 | Y57H | 62.5 |
| 11 | Hy1-A1-M1 | Y57H, P80C, A160C | 62.4 |
| 12 | Hy1-A1-M2 | Y57H, G291C, Y362C | 61.5 |
| 13 | Hy1-A1-M3 | Y57H, L180C, A223C | 62.4 |
| 14 | Hy1-A1-M4 | Y57H, P6C, Y403C | 61.5 |
| 15 | Hy1-A1-M5 | Y57H, S36C, D426C | 62.0 |
| 16 | Hy1-A2 | W304H | 61.5 |
| 17 | Hy1-A2-M1 | W304H, P80C, A160C | 62.4 |
| 18 | Hy1-A2-M2 | W304H, G291C, Y362C | 61.5 |
| 19 | Hy1-A2-M3 | W304H, L180C, A223C | 62.4 |
| 20 | Hy1-A2-M4 | W304H, P6C, Y403C | 61.5 |
| 21 | Hy1-A2-M5 | W304H, S36C, D426C | 61.9 |
| 22 | Hy1-A3 | T306R | 61.8 |
| 23 | Hy1-A3-M1 | T306R, P80C, A160C | 62.7 |
| 24 | Hy1-A3-M2 | T306R, G291C, Y362C | 61.8 |
| 25 | Hy1-A3-M3 | T306R, L180C, A223C | 62.7 |
| 26 | Hy1-A3-M4 | T306R, P6C, Y403C | 61.8 |
| 27 | Hy1-A3-M5 | T306R, S36C, D426C | 62.3 |
| 28 | Hy1-A4 | L307K | 60.6 |
| 29 | Hy1-A4-M1 | L307K, P80C, A160C | 61.6 |
| 30 | Hy1-A4-M2 | L307K, G291C, Y362C | 60.7 |
| 31 | Hy1-A4-M3 | L307K, L180C, A223C | 61.5 |
| 32 | Hy1-A4-M4 | L307K, P6C, Y403C | 60.6 |
| 33 | Hy1-A4-M5 | L307K, S36C, D426C | 61.1 |
| 34 | Hy1-A5 | T306A | 61.6 |
| 35 | Hy1-A5-M1 | T306A, P80C, A160C | 62.5 |
| 36 | Hy1-A5-M2 | T306A, G291C, Y362C | 61.6 |
| 37 | Hy1-A5-M3 | T306A, L180C, A223C | 62.5 |
| 38 | Hy1-A5-M4 | T306A, P6C, Y403C | 61.6 |
| 39 | Hy1-A5-M5 | T306A, S36C, D426C | 62.0 |
| 40 | Hy2-M1 | P80C, A160C | 63.1 |
| 41 | Hy2-M2 | G291C, Y362C | 62.2 |
| 42 | Hy2-M3 | L180C, A223C | 63.0 |
| 43 | Hy2-M4 | P6C, Y403C | 62.1 |
| 44 | Hy2-M5 | S36C, D426C | 62.6 |
| 45 | Hy2-A1 | Y57H | 62.2 |
| 46 | Hy2-A1-M1 | Y57H, P80C, A160C | 63.1 |
| 47 | Hy2-A1-M2 | Y57H, G291C, Y362C | 62.2 |
| 48 | Hy2-A1-M3 | Y57H, L180C, A223C | 63.1 |
| 49 | Hy2-A1-M4 | Y57H, P6C, Y403C | 62.2 |
| 50 | Hy2-A1-M5 | Y57H, S36C, D426C | 62.6 |
| 51 | Hy2-A2 | W304H | 62.2 |
| 52 | Hy2-A2-M1 | W304H, P80C, A160C | 63.1 |
| 53 | Hy2-A2-M2 | W304H, G291C, Y362C | 62.2 |
| 54 | Hy2-A2-M3 | W304H, L180C, A223C | 63.0 |
| 55 | Hy2-A2-M4 | W304H, P6C, Y403C | 62.2 |
| 56 | Hy2-A2-M5 | W304H, S36C, D426C | 62.6 |
| 57 | Hy2-A3 | T306R | 62.5 |
| 58 | Hy2-A3-M1 | T306R, P80C, A160C | 63.4 |
| 59 | Hy2-A3-M2 | T306R, G291C, Y362C | 62.5 |
| 60 | Hy2-A3-M3 | T306R, L180C, A223C | 63.4 |
| 61 | Hy2-A3-M4 | T306R, P6C, Y403C | 62.5 |
| 62 | Hy2-A3-M5 | T306R, S36C, D426C | 62.9 |
| 63 | Hy2-A4 | L307K | 61.3 |
| 64 | Hy2-A4-M1 | L307K, P80C, A160C | 62.2 |
| 65 | Hy2-A4-M2 | L307K, G291C, Y362C | 61.3 |
| 66 | Hy2-A4-M3 | L307K, L180C, A223C | 62.2 |
| 67 | Hy2-A4-M4 | L307K, P6C, Y403C | 61.3 |
| 68 | Hy2-A4-M5 | L307K, S36C, D426C | 61.8 |
| 69 | Hy2-A5 | T306A | 62.3 |
| 70 | Hy2-A5-M1 | T306A, P80C, A160C | 63.2 |
| 71 | Hy2-A5-M2 | T306A, G291C, Y362C | 62.3 |
| 72 | Hy2-A5-M3 | T306A, L180C, A223C | 63.2 |
| 73 | Hy2-A5-M4 | T306A, P6C, Y403C | 62.3 |
| 74 | Hy2-A5-M5 | T306A, S36C, D426C | 62.7 |
| 75 | Hy3-M1 | P80C, A160C | 63.5 |
| 76 | Hy3-M2 | G291C, Y362C | 62.6 |
| 77 | Hy3-M3 | L180C, A223C | 63.5 |
| 78 | Hy3-M4 | P6C, Y403C | 62.6 |
| 79 | Hy3-M5 | S36C, D426C | 63.0 |
| 80 | Hy3-A1 | Y57H | 62.6 |
| 81 | Hy3-A1-M1 | Y57H, P80C, A160C | 63.6 |
| 82 | Hy3-A1-M2 | Y57H, G291C, Y362C | 62.7 |
| 83 | Hy3-A1-M3 | Y57H, L180C, A223C | 63.5 |
| 84 | Hy3-A1-M4 | Y57H, P6C, Y403C | 62.6 |
| 85 | Hy3-A1-M5 | Y57H, S36C, D426C | 63.1 |
| 86 | Hy3-A2 | W304H | 62.6 |
| 87 | Hy3-A2-M1 | W304H, P80C, A160C | 63.5 |
| 88 | Hy3-A2-M2 | W304H, G291C, Y362C | 62.6 |
| 89 | Hy3-A2-M3 | W304H, L180C, A223C | 63.5 |
| 90 | Hy3-A2-M4 | W304H, P6C, Y403C | 62.6 |
| 91 | Hy3-A2-M5 | W304H, S36C, D426C | 63.0 |
| 92 | Hy3-A3 | T306R | 62.9 |
| 93 | Hy3-A3-M1 | T306R, P80C, A160C | 63.9 |
| 94 | Hy3-A3-M2 | T306R, G291C, Y362C | 63.0 |
| 95 | Hy3-A3-M3 | T306R, L180C, A223C | 63.8 |
| 96 | Hy3-A3-M4 | T306R, P6C, Y403C | 62.9 |
| 97 | Hy3-A3-M5 | T306R, S36C, D426C | 63.4 |
| 98 | Hy3-A4 | L307K | 61.8 |
| 99 | Hy3-A4-M1 | L307K, P80C, A160C | 62.7 |
| 100 | Hy3-A4-M2 | L307K, G291C, Y362C | 61.8 |
| 101 | Hy3-A4-M3 | L307K, L180C, A223C | 62.7 |
| 102 | Hy3-A4-M4 | L307K, P6C, Y403C | 61.8 |
| 103 | Hy3-A4-M5 | L307K, S36C, D426C | 62.2 |
| 104 | Hy3-A5 | T306A | 62.7 |
| 105 | Hy3-A5-M1 | T306A, P80C, A160C | 63.6 |
| 106 | Hy3-A5-M2 | T306A, G291C, Y362C | 62.7 |
| 107 | Hy3-A5-M3 | T306A, L180C, A223C | 63.6 |
| 108 | Hy3-A5-M4 | T306A, P6C, Y403C | 62.7 |
| 109 | Hy3-A5-M5 | T306A, S36C, D426C | 63.2 |

### Example 5. Stability Evaluation of Hyaluronidase PH-20 Variants (37 °C, 3 Days)

To confirm the aqueous stability of hyaluronidase PH-20 variants with active site mutations or newly introduced internal disulfide bonds by cysteine introduction, a 37 °C incubation test was conducted. Wild-type PH-20 and variants were dissolved in PBS (phosphate buffer saline), which is most similar to human conditions, at 0.5 mg/ml, incubated in a 37 °C water bath, and sampled daily. The samples were centrifuged at 13000 rpm for 15 minutes at 4 °C to obtain supernatant, and enzyme activity evaluation was conducted.

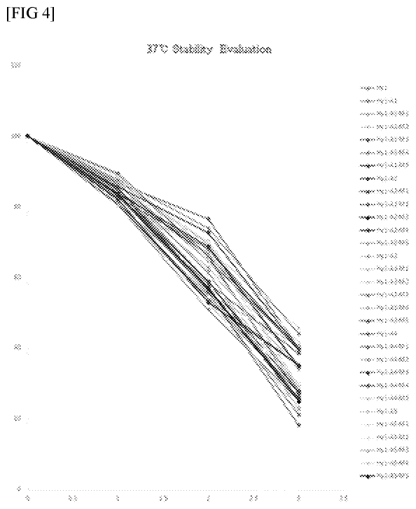

As a result, the stability of hyaluronidase at 37 °C was as shown in Table 5, and some of them are shown in FIG. 4.

TABLE 5
Stability Evaluation (37 °C, days)

| SEQ ID NO. | Name | 0 | 1 | 2 | 3 |
|---|---|---|---|---|---|
| 2 | Hy1 | 100 | 80.48 | 50.81 | 25.21 |
| 3 | Hy2 | 100 | 87.69 | 61.01 | 36.50 |
| 4 | Hy3 | 100 | 89.28 | 62.51 | 41.65 |
| 5 | Hy1-M1 | 100 | 85.61 | 72.65 | 39.66 |
| 6 | Hy1-M2 | 100 | 85.28 | 56.51 | 25.76 |
| 7 | Hy1-M3 | 100 | 82.68 | 68.07 | 39.42 |
| 8 | Hy1-M4 | 100 | 83.44 | 56.87 | 24.84 |
| 9 | Hy1-M5 | 100 | 83.69 | 68.72 | 34.75 |
| 10 | Hy1-A1 | 100 | 82.09 | 54.31 | 27.31 |
| 11 | Hy1-A1-M1 | 100 | 83.67 | 55.39 | 40.47 |
| 12 | Hy1-A1-M2 | 100 | 87.34 | 61.00 | 27.82 |
| 13 | Hy1-A1-M3 | 100 | 86.78 | 76.53 | 40.01 |
| 14 | Hy1-A1-M4 | 100 | 80.41 | 53.70 | 27.14 |
| 15 | Hy1-A1-M5 | 100 | 80.73 | 54.07 | 35.10 |
| 16 | Hy1-A2 | 100 | 83.91 | 57.15 | 25.73 |
| 17 | Hy1-A2-M1 | 100 | 86.52 | 64.76 | 40.21 |
| 18 | Hy1-A2-M2 | 100 | 89.05 | 66.32 | 26.77 |
| 19 | Hy1-A2-M3 | 100 | 84.45 | 66.25 | 39.60 |
| 20 | Hy1-A2-M4 | 100 | 85.06 | 58.87 | 25.42 |
| 21 | Hy1-A2-M5 | 100 | 86.07 | 66.77 | 35.06 |
| 22 | Hy1-A3 | 100 | 87.22 | 69.50 | 34.07 |
| 23 | Hy1-A3-M1 | 100 | 88.97 | 67.84 | 45.61 |
| 24 | Hy1-A3-M2 | 100 | 85.63 | 70.25 | 34.46 |
| 25 | Hy1-A3-M3 | 100 | 87.69 | 73.88 | 44.04 |
| 26 | Hy1-A3-M4 | 100 | 84.31 | 62.70 | 34.00 |
| 27 | Hy1-A3-M5 | 100 | 80.85 | 58.26 | 38.49 |
| 28 | Hy1-A4 | 100 | 80.55 | 54.98 | 21.12 |
| 29 | Hy1-A4-M1 | 100 | 89.50 | 65.61 | 28.09 |
| 30 | Hy1-A4-M2 | 100 | 86.39 | 61.56 | 22.78 |
| 31 | Hy1-A4-M3 | 100 | 83.87 | 58.75 | 27.55 |
| 32 | Hy1-A4-M4 | 100 | 83.57 | 56.08 | 18.10 |
| 33 | Hy1-A4-M5 | 100 | 80.35 | 52.70 | 23.35 |
| 34 | Hy1-A5 | 100 | 81.89 | 68.89 | 29.36 |
| 35 | Hy1-A5-M1 | 100 | 88.41 | 69.82 | 41.55 |
| 36 | Hy1-A5-M2 | 100 | 81.89 | 54.50 | 30.06 |
| 37 | Hy1-A5-M3 | 100 | 82.43 | 66.50 | 40.51 |
| 38 | Hy1-A5-M4 | 100 | 85.37 | 64.83 | 29.07 |
| 39 | Hy1-A5-M5 | 100 | 82.01 | 52.85 | 35.14 |
| 40 | Hy2-M1 | 100 | 88.00 | 58.89 | 48.21 |
| 41 | Hy2-M2 | 100 | 82.78 | 66.80 | 36.81 |
| 42 | Hy2-M3 | 100 | 86.39 | 68.61 | 46.78 |
| 43 | Hy2-M4 | 100 | 82.53 | 71.40 | 36.45 |
| 44 | Hy2-M5 | 100 | 83.83 | 70.48 | 41.78 |
| 45 | Hy2-A1 | 100 | 81.61 | 67.57 | 37.12 |
| 46 | Hy2-A1-M1 | 100 | 81.58 | 68.28 | 49.46 |
| 47 | Hy2-A1-M2 | 100 | 86.53 | 67.23 | 37.44 |
| 48 | Hy2-A1-M3 | 100 | 83.60 | 73.36 | 49.20 |
| 49 | Hy2-A1-M4 | 100 | 80.07 | 65.75 | 37.27 |
| 50 | Hy2-A1-M5 | 100 | 86.67 | 67.02 | 43.43 |
| 51 | Hy2-A2 | 100 | 86.07 | 61.65 | 36.68 |
| 52 | Hy2-A2-M1 | 100 | 87.04 | 65.50 | 49.12 |
| 53 | Hy2-A2-M2 | 100 | 84.50 | 74.03 | 36.91 |
| 54 | Hy2-A2-M3 | 100 | 80.42 | 70.07 | 47.42 |
| 55 | Hy2-A2-M4 | 100 | 86.10 | 59.63 | 36.55 |
| 56 | Hy2-A2-M5 | 100 | 86.42 | 72.89 | 42.75 |
| 57 | Hy2-A3 | 100 | 88.00 | 58.81 | 41.13 |
| 58 | Hy2-A3-M1 | 100 | 85.91 | 71.99 | 54.37 |
| 59 | Hy2-A3-M2 | 100 | 81.01 | 65.97 | 41.52 |
| 60 | Hy2-A3-M3 | 100 | 83.05 | 72.61 | 53.45 |
| 61 | Hy2-A3-M4 | 100 | 86.88 | 57.86 | 40.83 |
| 62 | Hy2-A3-M5 | 100 | 83.75 | 58.07 | 46.67 |
| 63 | Hy2-A4 | 100 | 81.17 | 57.26 | 24.21 |
| 64 | Hy2-A4-M1 | 100 | 81.67 | 56.20 | 37.49 |
| 65 | Hy2-A4-M2 | 100 | 88.80 | 61.01 | 24.32 |
| 66 | Hy2-A4-M3 | 100 | 87.52 | 70.61 | 37.38 |
| 67 | Hy2-A4-M4 | 100 | 84.99 | 62.12 | 24.05 |
| 68 | Hy2-A4-M5 | 100 | 83.86 | 71.26 | 32.08 |
| 69 | Hy2-A5 | 100 | 84.60 | 63.21 | 37.75 |
| 70 | Hy2-A5-M1 | 100 | 88.25 | 75.68 | 50.71 |
| 71 | Hy2-A5-M2 | 100 | 88.25 | 60.25 | 39.04 |
| 72 | Hy2-A5-M3 | 100 | 80.75 | 65.90 | 50.49 |
| 73 | Hy2-A5-M4 | 100 | 80.72 | 60.27 | 37.60 |
| 74 | Hy2-A5-M5 | 100 | 85.64 | 60.32 | 44.95 |
| 75 | Hy3-M1 | 100 | 89.73 | 67.50 | 56.24 |
| 76 | Hy3-M2 | 100 | 86.22 | 67.80 | 42.90 |
| 77 | Hy3-M3 | 100 | 84.18 | 71.08 | 55.60 |
| 78 | Hy3-M4 | 100 | 85.24 | 68.65 | 41.65 |
| 79 | Hy3-M5 | 100 | 81.90 | 59.14 | 47.07 |
| 80 | Hy3-A1 | 100 | 84.82 | 56.67 | 43.36 |
| 81 | Hy3-A1-M1 | 100 | 85.43 | 74.09 | 57.92 |
| 82 | Hy3-A1-M2 | 100 | 86.13 | 75.37 | 43.86 |
| 83 | Hy3-A1-M3 | 100 | 87.19 | 73.18 | 56.56 |
| 84 | Hy3-A1-M4 | 100 | 81.06 | 69.62 | 43.30 |
| 85 | Hy3-A1-M5 | 100 | 85.61 | 67.05 | 48.64 |
| 86 | Hy3-A2 | 100 | 83.77 | 73.28 | 42.50 |
| 87 | Hy3-A2-M1 | 100 | 84.73 | 68.04 | 57.56 |
| 88 | Hy3-A2-M2 | 100 | 81.95 | 56.93 | 43.13 |
| 89 | Hy3-A2-M3 | 100 | 84.28 | 71.49 | 56.11 |
| 90 | Hy3-A2-M4 | 100 | 81.88 | 63.12 | 41.90 |
| 91 | Hy3-A2-M5 | 100 | 80.84 | 59.79 | 47.80 |
| 92 | Hy3-A3 | 100 | 86.68 | 74.11 | 46.55 |
| 93 | Hy3-A3-M1 | 100 | 84.75 | 74.23 | 62.19 |
| 94 | Hy3-A3-M2 | 100 | 81.89 | 58.04 | 46.69 |
| 95 | Hy3-A3-M3 | 100 | 84.70 | 72.61 | 60.98 |
| 96 | Hy3-A3-M4 | 100 | 89.17 | 70.94 | 45.83 |
| 97 | Hy3-A3-M5 | 100 | 83.26 | 70.39 | 53.02 |
| 98 | Hy3-A4 | 100 | 81.35 | 53.16 | 31.96 |
| 99 | Hy3-A4-M1 | 100 | 88.81 | 72.76 | 43.92 |
| 100 | Hy3-A4-M2 | 100 | 80.85 | 67.87 | 33.12 |
| 101 | Hy3-A4-M3 | 100 | 83.60 | 70.64 | 43.70 |
| 102 | Hy3-A4-M4 | 100 | 82.35 | 54.63 | 30.68 |
| 103 | Hy3-A4-M5 | 100 | 89.32 | 72.54 | 37.34 |
| 104 | Hy3-A5 | 100 | 88.53 | 59.91 | 44.82 |
| 105 | Hy3-A5-M1 | 100 | 84.09 | 72.30 | 59.21 |
| 106 | Hy3-A5-M2 | 100 | 83.28 | 66.25 | 45.53 |
| 107 | Hy3-A5-M3 | 100 | 80.52 | 70.10 | 58.95 |
| 108 | Hy3-A5-M4 | 100 | 82.64 | 72.10 | 44.33 |
| 109 | Hy3-A5-M5 | 100 | 84.20 | 60.88 | 50.36 |

### Example 6. Stability Evaluation of Hyaluronidase PH-20 Variants (50C, 3 Hours)

To confirm the aqueous stability of hyaluronidase PH-20 variants with active site mutations or newly introduced internal disulfide bonds by cysteine introduction, a 50 °C incubation test was conducted. Wild-type PH-20 and variants were dissolved in PBS (phosphate buffer saline), which is most similar to human conditions, at 0.5 mg/ml, incubated in a 50 °C water bath, and sampled hourly. The samples were centrifuged at 13000 rpm for 15 minutes at 4 °C to obtain supernatant, and enzyme activity evaluation was conducted.

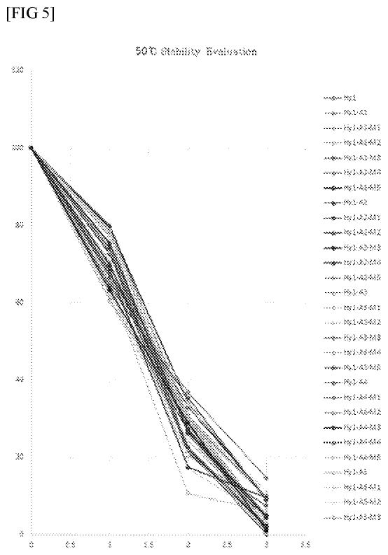

As a result, the stability of hyaluronidase at 50 °C was as shown in Table 6, and some of them are shown in FIG. 5.

TABLE 6
Stability Evaluation (50 °C, hours)

| SEQ ID NO. | Name | 0 | 1 | 2 | 3 |
|---|---|---|---|---|---|
| 2 | Hy1 | 100 | 77.19 | 22.46 | 1.02 |
| 3 | Hy2 | 100 | 75.68 | 31.29 | 5.16 |
| 4 | Hy3 | 100 | 78.62 | 34.87 | 10.91 |
| 5 | Hy1-M1 | 100 | 68.31 | 17.33 | 9.44 |
| 6 | Hy1-M2 | 100 | 69.79 | 22.45 | 2.15 |
| 7 | Hy1-M3 | 100 | 64.28 | 35.29 | 8.87 |
| 8 | Hy1-M4 | 100 | 63.25 | 28.83 | 1.01 |
| 9 | Hy1-M5 | 100 | 75.06 | 26.31 | 4.32 |
| 10 | Hy1-A1 | 100 | 60.72 | 28.39 | 2.29 |
| 11 | Hy1-A1-M1 | 100 | 78.99 | 30.71 | 9.82 |
| 12 | Hy1-A1-M2 | 100 | 77.48 | 28.31 | 2.32 |
| 13 | Hy1-A1-M3 | 100 | 72.58 | 29.59 | 9.57 |
| 14 | Hy1-A1-M4 | 100 | 75.31 | 27.48 | 2.28 |
| 15 | Hy1-A1-M5 | 100 | 72.74 | 21.16 | 4.65 |
| 16 | Hy1-A2 | 100 | 75.11 | 22.42 | 1.78 |
| 17 | Hy1-A2-M1 | 100 | 67.24 | 32.88 | 9.59 |
| 18 | Hy1-A2-M2 | 100 | 79.85 | 29.75 | 2.22 |
| 19 | Hy1-A2-M3 | 100 | 79.36 | 33.90 | 9.05 |
| 20 | Hy1-A2-M4 | 100 | 62.48 | 26.64 | 1.68 |
| 21 | Hy1-A2-M5 | 100 | 62.77 | 29.09 | 4.56 |
| 22 | Hy1-A3 | 100 | 73.19 | 30.79 | 4.21 |
| 23 | Hy1-A3-M1 | 100 | 64.28 | 10.55 | 6.12 |
| 24 | Hy1-A3-M2 | 100 | 74.95 | 17.14 | 4.24 |
| 25 | Hy1-A3-M3 | 100 | 65.10 | 36.72 | 14.61 |
| 26 | Hy1-A3-M4 | 100 | 69.82 | 30.18 | 4.18 |
| 27 | Hy1-A3-M5 | 100 | 64.47 | 20.42 | 7.47 |
| 28 | Hy1-A4 | 100 | 68.02 | 28.88 | 0.24 |
| 29 | Hy1-A4-M1 | 100 | 69.68 | 29.11 | 2.45 |
| 30 | Hy1-A4-M2 | 100 | 78.41 | 26.11 | 0.27 |
| 31 | Hy1-A4-M3 | 100 | 73.93 | 20.83 | 2.29 |
| 32 | Hy1-A4-M4 | 100 | 66.42 | 22.06 | 0.09 |
| 33 | Hy1-A4-M5 | 100 | 62.05 | 29.87 | 0.42 |
| 34 | Hy1-A5 | 100 | 70.59 | 22.35 | 3.04 |
| 35 | Hy1-A5-M1 | 100 | 76.68 | 33.66 | 10.84 |
| 36 | Hy1-A5-M2 | 100 | 64.69 | 20.52 | 3.16 |
| 37 | Hy1-A5-M3 | 100 | 78.78 | 30.00 | 9.86 |
| 38 | Hy1-A5-M4 | 100 | 60.24 | 24.30 | 3.01 |
| 39 | Hy1-A5-M5 | 100 | 69.53 | 27.08 | 4.88 |
| 40 | Hy2-M1 | 100 | 71.40 | 39.45 | 18.63 |
| 41 | Hy2-M2 | 100 | 65.09 | 20.49 | 5.96 |
| 42 | Hy2-M3 | 100 | 72.04 | 43.99 | 17.84 |
| 43 | Hy2-M4 | 100 | 74.50 | 17.36 | 5.13 |
| 44 | Hy2-M5 | 100 | 63.60 | 12.62 | 11.09 |
| 45 | Hy2-A1 | 100 | 60.84 | 28.70 | 6.02 |
| 46 | Hy2-A1-M1 | 100 | 79.55 | 50.09 | 21.00 |
| 47 | Hy2-A1-M2 | 100 | 68.23 | 35.99 | 6.41 |
| 48 | Hy2-A1-M3 | 100 | 66.87 | 9.50 | 0.02 |
| 49 | Hy2-A1-M4 | 100 | 63.06 | 28.91 | 6.22 |
| 50 | Hy2-A1-M5 | 100 | 71.71 | 43.95 | 14.01 |
| 51 | Hy2-A2 | 100 | 71.18 | 31.19 | 5.6 |
| 52 | Hy2-A2-M1 | 100 | 76.82 | 48.00 | 19.83 |
| 53 | Hy2-A2-M2 | 100 | 70.03 | 20.93 | 5.97 |
| 54 | Hy2-A2-M3 | 100 | 63.47 | 41.05 | 18.26 |
| 55 | Hy2-A2-M4 | 100 | 70.24 | 22.03 | 5.59 |
| 56 | Hy2-A2-M5 | 100 | 63.16 | 16.61 | 11.91 |
| 57 | Hy2-A3 | 100 | 60.35 | 36.71 | 9.97 |
| 58 | Hy2-A3-M1 | 100 | 76.20 | 44.99 | 24.85 |
| 59 | Hy2-A3-M2 | 100 | 76.62 | 18.79 | 10.81 |
| 60 | Hy2-A3-M3 | 100 | 69.41 | 44.93 | 24.68 |
| 61 | Hy2-A3-M4 | 100 | 60.50 | 11.31 | 9.88 |
| 62 | Hy2-A3-M5 | 100 | 70.39 | 11.80 | 7.28 |
| 63 | Hy2-A4 | 100 | 74.70 | 24.30 | 0.61 |
| 64 | Hy2-A4-M1 | 100 | 64.63 | 33.23 | 6.51 |
| 65 | Hy2-A4-M2 | 100 | 62.39 | 25.42 | 0.72 |
| 66 | Hy2-A4-M3 | 100 | 74.54 | 36.09 | 6.38 |
| 67 | Hy2-A4-M4 | 100 | 74.33 | 19.80 | 0.56 |
| 68 | Hy2-A4-M5 | 100 | 60.47 | 19.97 | 4.11 |
| 69 | Hy2-A5 | 100 | 62.63 | 19.95 | 7.38 |
| 70 | Hy2-A5-M1 | 100 | 76.30 | 46.34 | 23.28 |
| 71 | Hy2-A5-M2 | 100 | 62.64 | 30.89 | 8.06 |
| 72 | Hy2-A5-M3 | 100 | 78.29 | 45.37 | 21.45 |
| 73 | Hy2-A5-M4 | 100 | 64.71 | 34.17 | 7.03 |
| 74 | Hy2-A5-M5 | 100 | 65.37 | 41.20 | 15.76 |
| 75 | Hy3-M1 | 100 | 68.08 | 46.46 | 25.20 |
| 76 | Hy3-M2 | 100 | 68.42 | 12.18 | 11.99 |
| 77 | Hy3-M3 | 100 | 74.80 | 51.42 | 24.88 |
| 78 | Hy3-M4 | 100 | 63.52 | 16.94 | 10.90 |
| 79 | Hy3-M5 | 100 | 61.29 | 5.53 | 0.07 |
| 80 | Hy3-A1 | 100 | 60.70 | 38.16 | 13.60 |
| 81 | Hy3-A1-M1 | 100 | 75.75 | 54.52 | 29.60 |
| 82 | Hy3-A1-M2 | 100 | 79.92 | 34.68 | 14.17 |
| 83 | Hy3-A1-M3 | 100 | 75.59 | 46.67 | 26.46 |
| 84 | Hy3-A1-M4 | 100 | 71.15 | 35.47 | 12.77 |
| 85 | Hy3-A1-M5 | 100 | 69.77 | 45.68 | 19.32 |
| 86 | Hy3-A2 | 100 | 70.96 | 34.45 | 11.47 |
| 87 | Hy3-A2-M1 | 100 | 71.80 | 49.67 | 26.54 |
| 88 | Hy3-A2-M2 | 100 | 77.51 | 34.66 | 12.03 |
| 89 | Hy3-A2-M3 | 100 | 66.46 | 45.77 | 25.11 |
| 90 | Hy3-A2-M4 | 100 | 76.30 | 34.99 | 11.25 |
| 91 | Hy3-A2-M5 | 100 | 78.47 | 41.82 | 18.61 |
| 92 | Hy3-A3 | 100 | 75.38 | 38.86 | 17.17 |
| 93 | Hy3-A3-M1 | 100 | 79.30 | 57.77 | 36.75 |
| 94 | Hy3-A3-M2 | 100 | 70.96 | 43.37 | 17.53 |
| 95 | Hy3-A3-M3 | 100 | 79.68 | 57.52 | 34.75 |
| 96 | Hy3-A3-M4 | 100 | 79.08 | 44.42 | 17.11 |
| 97 | Hy3-A3-M5 | 100 | 74.59 | 47.77 | 23.48 |
| 98 | Hy3-A4 | 100 | 74.86 | 18.99 | 3.74 |
| 99 | Hy3-A4-M1 | 100 | 73.79 | 15.18 | 14.34 |
| 100 | Hy3-A4-M2 | 100 | 60.70 | 30.19 | 4.12 |
| 101 | Hy3-A4-M3 | 100 | 63.06 | 40.08 | 14.05 |
| 102 | Hy3-A4-M4 | 100 | 72.83 | 23.33 | 3.32 |
| 103 | Hy3-A4-M5 | 100 | 79.10 | 29.74 | 6.31 |
| 104 | Hy3-A5 | 100 | 68.82 | 9.58 | 4.95 |
| 105 | Hy3-A5-M1 | 100 | 78.27 | 53.86 | 33.03 |
| 106 | Hy3-A5-M2 | 100 | 79.88 | 45.17 | 15.96 |
| 107 | Hy3-A5-M3 | 100 | 77.69 | 55.85 | 30.37 |
| 108 | Hy3-A5-M4 | 100 | 64.52 | 42.35 | 14.65 |
| 109 | Hy3-A5-M5 | 100 | 62.76 | 6.59 | 1.06 |

### INDUSTRIAL APPLICABILITY

As described above, the novel hyaluronidase PH-20 variant according to the present invention has higher stability and superior activity compared to the wild-type human hyaluronidase PH-20, thereby maximizing the therapeutic effect of drugs used in combination with it, and thus has high industrial applicability.

1. A hyaluronidase PH-20 variant selected from the group consisting of SEQ ID NO: 2, SEQ ID NO: 3, and SEQ ID NO: 4.

2. The hyaluronidase PH-20 variant according to claim 1, further comprising a substitution of any amino acid residue selected from the group consisting of i) Y57H, ii) W304H, iii) T306R, vi) L307K, and v) T306A.

3. The hyaluronidase PH-20 variant according to claim 1, further comprising a substitution of any amino acid residue selected from the group consisting of i) Y57H, ii) W304H, iii) T306R, and vi) L307K.

4. The hyaluronidase PH-20 variant according to claim 1, further comprising a substitution of any amino acid residue selected from the group consisting of i) P80C and A160C, ii) G291C and Y362C, iii) L180C and A223C, vi) P6C and Y403C, and v) S36C and D426C.

5. The hyaluronidase PH-20 variant according to claim 1, further comprising a substitution of any amino acid residue selected from the group consisting of i) G291C and Y362C, ii) L180C and A223C, iii) P6C and Y403C, and vi) S36C and D426C.

6. The hyaluronidase PH-20 variant according to claim 2, further comprising a substitution of any amino acid residue selected from the group consisting of i) P80C and A160C, ii) G291C and Y362C, iii) L180C and A223C, vi) P6C and Y403C, and v) S36C and D426C.

7. A polynucleotide encoding the hyaluronidase PH-20 variant according to claim 1.

8. An expression vector comprising the polynucleotide of claim 7.

9. A host cell line comprising the expression vector of claim 8.

10. A method for producing a hyaluronidase PH-20 variant, comprising culturing the host cell line according to claim 9.

11. A pharmaceutical composition for preventing or treating a disease, comprising:
a) a drug; and
b) the hyaluronidase PH-20 variant according to claim 1.

12. The pharmaceutical composition according to claim 11, wherein the drug is a protein drug, small molecule, aptamer, RNAi, antisense or cell therapeutic agent.

13. The pharmaceutical composition according to claim 11, wherein the drug is selected from the group consisting of a chemotherapeutic agent, analgesic, anti-inflammatory agent, antimicrobial agent, antibacterial agent, anti-trichomonal agent, anti-Parkinson's agent, antimalarial, anticonvulsant, antidepressant, antiarthritic agent, antifungal agent, antihypertensive, antipyretic, antiparasitic agent, antihistamine, alpha-adrenergic agonist, alpha blocker, anesthetic, bronchodilator, biocide, bactericide, beta-adrenergic blocking agent, calcium channel blocker, cardiovascular drug, contraceptive, decongestant, diuretic, depressant, diagnostic agent, electrolyte, hypnotic, hormones, hyperglycemic agent, muscle relaxant, muscle contractant, ophthalmic agent, parasympathomimetic agent, sympathomimetic agent, antiviral agent, vitamin, non-steroidal anti-inflammatory agent, angiotensin converting enzyme inhibitor, and sedative.

14. The pharmaceutical composition according to claim 11, wherein the drug is an antibody, soluble receptor, or soluble receptor-Fc fusion protein.

15. The pharmaceutical composition according to claim 14, wherein the antibody binds to one or more antigens selected from the group consisting of 4-11BB, integrin, amyloid beta, angiopoietin, angiopoietin-like 3, B-cell activating factor (BAFF), B7-H3, complement 5, CCR4, CD3, CD4, CD6, CD11a, CD19, CD20, CD22, CD30, CD33, CD38, CD52, CD62, CD79b, CD80, CGRP, Claudin-18, complement factor D, CTLA4, DLL3, EGF receptor, hemophilia factor, Fc receptor, FGF23, folate receptor, GD2, GM-CSF, HER2, HER3, interferon receptor, interferon gamma, IgE, IGF-1 receptor, interleukin 1, interleukin 2 receptor, interleukin 4 receptor, interleukin 5, interleukin 5 receptor, interleukin 6, interleukin 6 receptor, interleukin 7, interleukin 12/23, interleukin 13, interleukin 17A, interleukin 17 receptor A, interleukin 31 receptor, interleukin 36 receptor, LAG3, LFA3, NGF, PVSK9, PD-1, PD-L1, RANK-L, SLAMF7, Tissue factor, TNF, VEGF, and vWF.

16. The pharmaceutical composition according to claim 14, wherein the antibody is selected from one or more of Utomilumab, Natalizumab, Etrolizumab, Vedolizumab, Bimagrumab, Bapineuzumab, Crenezumab, Solanezumab, Aducanumab, Gantenerumab, AMG 780, MEDI 3617, Nesvacumab, Vanucizumab, Evinacumab, Tabalumab, Lanalumab, Belimumab, Omburtamab, Ravulizumab, Eculizumab, Mogamulizumab, Otelixizumab, Teplizumab, Muromonab, Tebentafusp, Blinatumomab, REGN1979, Ibalizumab, Zanolimumab, Itolizumab, Efalizumab, Inebilizumab, Tafasitamab, Loncastuximab tesirine, Ocrelizumab, Ublituximab, Obinutuzumab, Ofatumumab, Rituximab, Tositumomab, Ibritumomab tiuxetan, Epratuzumab, Inotuzumab ozogamicin, Moxetumomab pasudotox, Brentuximab vedotin, Vadastuximab talirine, Gemtuzumab ozogamicin, Daratumumab, Isatuximab, Alemtuzumab, Crizanlizumab, Polatuzumab vedotin, Galiximab, Eptinezumab, Fremanezumab, Galcanezumab, Erenumab, Zolbetuximab, Lampalizumab, Tremelimumab, Zalifrelimab, Ipilimumab, Rovalpituzumab tesirine, Cetuximab, Depatuxizumab, Zalutumumab, Necitumumab, Panitumumab, Emicizumab, Nipocalimab, Rozanolixizumab, Burosumab, Farletuzumab, Mirvetuximab soravtansine, Dinutuximab, Naxitamab, Otilimab, Margetuximab, Pertuzumab, Trastuzumab, Trastuzumab deruxtecan, Trastuzumab emtansine, Trastuzumab duocarmazine, Patritumab, Anifrolumab, Emapalumab, Ligelizumab, Omalizumab, Dalotuzumab, Figitumumab, Teprotumumab, Gebokizumab, Canakinumab, Daclizumab, Basiliximab, Dupilumab, Mepolizumab, Reslizumab, Benralizumab, Clazakizumab, Olokizumab, Sirukumab, Siltuximab, Sarilumab, Satralizumab, Tocilizumab, REGN88, Secukinumab, Ustekinumab, Briakinumab, Lebrikizumab, Tralokinumab, Ixekizumab, Bimekizumab, Brodalumab, Brazikumab, Guselkumab, Risankizumab, Tildrakizumab, Mirikizumab, Nemolizumab, Spesolimab, Relatlimab, Narsoplimab, Fasinumab, Tanezumab, Alirocumab, Evolocumab, Bococizumab, Lambrolizumab, Balstilimab, Camrelizumab, Cemiplimab, Dostarlimab, Prolgolimab, Sintilimab, Spartalizumab, Tislelizumab, Pembrolizumab, Nivolumab, Atezolizumab, Avelumab, Envafolimab, Durvalumab, Bintrafusp alpha, Denosumab, Elotuzumab, Concizumab, Marstacimab, Infliximab, Adalimumab, Golimumab, Certolizumab pegol, Ozoralizumab, Brolucizumab, Ranibizumab, Bevacizumab, Faricimab, Ramucirumab, and Caplacizumab.

17. The pharmaceutical composition according to claim 14, wherein the soluble receptor included in the soluble receptor or soluble receptor-Fc fusion protein is selected from the group consisting of TNF-α soluble receptor, VEGF soluble receptor, CTLA-4, interleukin 1 soluble receptor, and LFA3 soluble receptor.

18. The pharmaceutical composition according to claim 17, wherein the soluble receptor-Fc fusion protein is selected from the group consisting of Etanercept, Aflibercept, Abatacept, Belatacept, Rilonacept, and Alefacept.

19. The pharmaceutical composition according to claim 11, further comprising one or more selected from the group consisting of a buffer, stabilizer, and surfactant.

20. The pharmaceutical composition according to claim 19, wherein
the buffer is one or more buffers selected from the group consisting of malate, formate, citrate, acetate, propionate, pyridine, piperazine, cacodylate, succinate, 2-(N-morpholino)ethanesulfonic acid (MES), histidine, Tris, bis-Tris, phosphate, ethanolamine, carbonate, piperazine-N,N′-bis(2-ethanesulfonic acid) (PIPES), imidazole, BIS-TRIS propane, N,N-bis(2-hydroxyethyl)-2-aminoethanesulfonic acid (BES), 3-(N-morpholino) propanesulfonic acid (MOPS), Hydroxyethyl piperazine Ethane Sulfonic acid (HEPES), pyrophosphate, and triethanolamine;
the stabilizer is one or more selected from the group consisting of carbohydrates, sugars or their hydrates, sugar alcohols or their hydrates, and amino acids; and
the surfactant is one or more nonionic surfactants selected from the group consisting of polyoxyethylene-sorbitan fatty acid, polyethylene-polypropylene glycol, polyoxyethylene stearate, polyoxyethylene alkyl ether, polyoxyethylene-polyoxypropylene copolymer, and sodium dodecyl sulfate (SDS).

21. The pharmaceutical composition according to claim 20, wherein
the carbohydrate, sugar or sugar alcohol is one or more selected from the group consisting of trehalose or its hydrate, sucrose, saccharin, glycerol, erythritol, threitol, xylitol, arabitol, ribitol, mannitol, sorbitol, galactitol, fucitol, iditol, inositol, volemitol, isomalt, maltitol, polyglycitol, cyclodextrin, Hydroxypropyl Beta-cyclodextrin, and glucose; and
the amino acid is one or more selected from the group consisting of glutamine, glutamic acid, glycine, lysine, lysyllysine, leucine, methionine, valine, serine, selenomethionine, citrulline, arginine, asparagine, aspartic acid, ornithine, isoleucine, taurine, theanine, threonine, tryptophan, tyrosine, phenylalanine, proline, pyrrolysine, histidine, and alanine.

22. The pharmaceutical composition according to claim 11, wherein the pharmaceutical composition is for subcutaneous administration.

23. Use of
a) a drug; and
b) the hyaluronidase PH-20 variant according to claim 1;
for manufacturing a pharmaceutical composition for treating a disease.

24. A method for treating a disease, comprising administering to a subject in need thereof an effective amount of a composition comprising as active ingredients:
a) a drug; and
b) the hyaluronidase PH-20 variant according to claim 1.
